(19) United States
(12) Patent Application Publication
(10) Pub. No.: US 2026/0034102 A1
(43) Pub. Date: Feb. 5, 2026

(54) HODGKIN LYMPHOMA THERAPY

(71) Applicant: Purdue Pharmaceutical Products L.P., Stamford, CT (US)

(72) Inventors: Thomas Mehrling, Riehen (CH); Rosaria De Filippi, Naples (IT); Antonio Pinto, Naples (IT)

(73) Assignee: Purdue Pharmaceutical Products L.P., Stamford, CT (US)

(21) Appl. No.: 19/355,530

(22) Filed: Oct. 10, 2025

Related U.S. Application Data
(63) Continuation of application No. 18/446,522, filed on Aug. 9, 2023, now Pat. No. 12,440,475.
Application No. 18/446,522 is a continuation of application No. 17/679,308, filed on Feb. 24, 2022, now Pat. No. 11,766,424.
Application No. 17/679,308 is a division of application No. 16/341,089, now Pat. No. 11,266,631.

(51) Int. Cl. A61K 31/4184; A61K 45/06; A61K 47/68; A61P 35/00

(52) U.S. Cl. CPC A61K 31/4184; A61K 47/68031; A61P 35/00; A61K 45/06

(57) ABSTRACT

There is provided a compound of formula I or a pharmacologically acceptable salt thereof for use in a method of treating Hodgkin lymphoma in a patient in need thereof comprising administering to said patient an effective amount of said compound of formula I or a pharmacologically acceptable salt thereof:

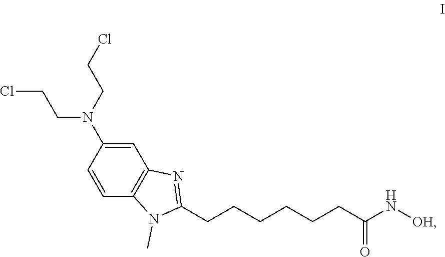 [SMILES: Cn1c(CCCCCCC(=O)NO)nc2cc(N(CCCl)CCCl)ccc21]

a combination of said compound of formula I or a pharmaceutically acceptable salt thereof with Brentuximab Vedotin and said combination for use in a method of treating Hodgkin lymphoma in a patient in need thereof comprising administering to said patient an effective amount of said combination.

### CROSS-REFERENCE TO RELATED APPLICATIONS

This application is a Continuation Application of U.S. application Ser. No. 18/446,522, filed on Aug. 9, 2023, which is a Continuation Application of U.S. application Ser. No. 17/679,308, filed on Feb. 24, 2022, now U.S. Pat. No. 11,766,424, issued on Sep. 26, 2023, which is a Divisional Application of U.S. application Ser. No. 16/341,089, filed on Apr. 11, 2019, now U.S. Pat. No. 11,266,631, issued on Mar. 8, 2022, which is a U.S. national stage filing under 35 U.S.C. § 371 of International Patent Application No. PCT/EP2016/074331, filed on Oct. 11, 2016. The entire contents of each of the aforementioned applications are incorporated herein by reference.

### TECHNICAL FIELD

The present invention relates to a method of treating Hodgkin lymphoma and to a combination and kit useful in a method of treating Hodgkin lymphoma.

### BACKGROUND TO THE INVENTION

Cancer is one of the most life threatening diseases. Cancer is a condition in which cells in a part of the body experience out-of-control growth. According to latest data from American Cancer Society, it is estimated there will be 1.69 million new cases of cancer in USA in 2016. Cancer is the second leading cause of death in the United States (second only to heart disease) and will claim more than 595,000 lives in 2016. In fact, it is estimated that 50% of all men and 33% of all women living in the United States will develop some type of cancer in their lifetime. Therefore cancer constitutes a major public health burden and represents a significant cost in the United States. These figures are reflected elsewhere across most countries globally, although the types of cancer and relative proportions of the population developing the cancers vary depending upon many different factors such including genetics and diet.

For decades surgery, chemotherapy, and radiation were the established treatments for various cancers. Patients usually receive a combination of these treatments depending upon the type and extent of their disease. But chemotherapy is the most important option for cancer patients when surgical treatment (i.e. the removal of diseased tissue) is impossible. While surgery is sometimes effective in removing tumours located at certain sites, for example, in the breast, colon, and skin, it cannot be used in the treatment of tumours located in other areas, such as the backbone, nor in the treatment of disseminated hematologic cancers include cancers of the blood and blood-forming tissues (such as the bone marrow). They include multiple myeloma, lymphoma and leukemia. Radiation therapy involves the exposure of living tissue to ionizing radiation causing death or damage to the exposed cells. Side effects from radiation therapy may be acute and temporary, while others may be irreversible. Chemotherapy involves the disruption of cell replication or cell metabolism. It is used most often in the treatment of breast, lung, and testicular cancer. One of the main causes of failure in this treatment of cancer is the development of drug resistance by the cancer cells, a serious problem that may lead to recurrence of disease or even death. Thus, more effective cancer treatments are needed.

Lymphoma is a cancer of the lymphatic system. There are two main types of lymphoma, namely Hodgkin lymphoma and non Hodgkin lymphoma.

Non Hodgkin lymphoma is the more common form of lymphoma. The lymphatic system runs throughout the body, and it is therefore possible to find non Hodgkin lymphoma in almost all parts of the body. In patients with non Hodgkin lymphoma, some of their white blood cells (lymphocytes) divide abnormally. They do not have any resting time like normal cells and they start to divide continuously, so too many are produced. They do not naturally die off as they usually do. These cells start to divide before they are fully mature and therefore cannot fight infection as normal white blood cells do. All the abnormal lymphocytes start to collect in the lymph nodes or other places such as the bone marrow or spleen. They can then grow into tumours and begin to cause problems within the lymphatic system or the organ in which they are growing. For example, if a lymphoma starts in the thyroid gland it can affect the normal production of thyroid hormones. There are many different types of non Hodgkin lymphoma. They can be classified in several different ways. One way is by the type of cell affected. In non Hodgkin lymphoma two types of lymphocyte can be affected-B cells and T cells. This is classified as B cell lymphoma or a T cell lymphoma. Most people with non Hodgkin lymphoma have B cell lymphomas. T cell lymphomas are more common in teenagers and young adults.

Hodgkin lymphoma can occur in both children and adults, but it is most commonly diagnosed in young adults between 20 and 34 years of age. Approximately 9,000 new cases are diagnosed in the US each year. The cells of Hodgkin lymphoma have a particular appearance under the microscope. These cells are called Reed Sternberg cells. Non Hodgkin lymphomas do not have Reed Sternberg cells. It is important for doctors to be able to tell the difference between Hodgkin lymphoma and non Hodgkin lymphoma cells as they are two different diseases. In Hodgkin lymphoma, it is cells in the lymph nodes that have become cancerous.

Hodgkin lymphoma has been divided into two main classifications: classical Hodgkin lymphoma, which accounts for 90 to 95 percent of cases, and nodular lymphocyte predominant Hodgkin lymphoma. The type of Hodgkin lymphoma a patient may affect their treatment choices.

Classical Hodgkin Lymphoma:

Nodular Sclerosis classical Hodgkin lymphoma is the most common subtype of classical Hodgkin lymphoma, accounting for 60 to 80 percent of all classical Hodgkin lymphoma cases. In nodular (knot-like) sclerosis classical Hodgkin lymphoma, the involved lymph nodes contain Reed Sternberg cells mixed with normal white blood cells. The lymph nodes often contain a lot of scar tissue, which is where the name nodular sclerosis (scarring) originates. The disease is more common in women than in men, and it usually affects adolescents and adults under the age of 50. The majority of patients are cured with current treatments.

Mixed cellularity classical Hodgkin lymphoma accounts for about 15 to 30 percent of all Hodgkin lymphoma cases. The disease is found more commonly in men than in women, and it primarily affects older adults. With this type of classical Hodgkin lymphoma, the lymph nodes contain many Reed Sternberg cells in addition to several other cell types. More advanced disease is usually present by the time this subtype is diagnosed.

Lymphocyte-Depletion classical Hodgkin lymphoma is rarely diagnosed. Abundant Reed Sternberg cells and few normal lymphocytes are present in the lymph nodes of patients with this subtype, which is aggressive and usually not diagnosed until it is widespread throughout the body.

Lymphocyte-Rich classical Hodgkin lymphoma accounts for less than five percent of Hodgkin lymphoma cases. The disease may be diffuse or nodular in form and is characterized by the presence of numerous normal-appearing lymphocytes and classic Reed Sternberg cells. This subtype of Hodgkin lymphoma is usually diagnosed at an early stage in adults and has a low relapse rate.

### Lymphocyte Predominant Hodgkin Lymphoma

Nodular Lymphocyte Predominant Hodgkin lymphoma accounts for five to 10 percent of all Hodgkin lymphoma cases. It affects men more often than women and is usually diagnosed before the age of 35. In nodular lymphocyte predominant Hodgkin lymphoma, most of the lymphocytes found in the lymph nodes are normal (not cancerous). Typical Reed Sternberg cells are usually not found in this subtype, but large, abnormal B cells (sometimes referred to as popcorn cells) can be seen as well as small B cells, which may be distributed in a nodular pattern within the tissues. This subtype is usually diagnosed at an early stage and is not very aggressive. In many ways, this form of Hodgkin lymphoma resembles indolent B-cell non Hodgkin lymphoma with late recurrences.

The % survival rate over 5 years in 2015 for patients with non Hodgkin lymphoma was 63%, while the survival rate for those with Hodgkin lymphoma over the same period was 83%. Over 80 percent of patients with Hodgkin lymphoma survive for five years, and many are cured. Most patients treated for Hodgkin lymphoma will receive some form of chemotherapy, and sometimes radiation therapy, as their first treatment. The recommended first-line therapy for Hodgkin lymphoma is ABVD (adriamycin, bleomycin, vinblastine, and dacarbazine) with or without radiation therapy or other agents, depending on the patient's type and stage of Hodgkin lymphoma as well as their overall health status. Other chemotherapy regimens adopted include mechlorethamine, vincristine, prednisone, and procarbazine. Brentuximab vedotin (Adcetris) was approved in 2011 by the U.S. Food and Drug Administration for the treatment of relapsed/refractory Hodgkin lymphoma after stem cell transplantation or after failure of two previous chemotherapy regimens in patients who are not eligible for stem cell transplantation.

In WO-A-2010/085377, the compound of formula I below is disclosed. It is a first-in-class dual-functional alkylating-HDACi fusion molecule which potently inhibits the HDAC pathway.

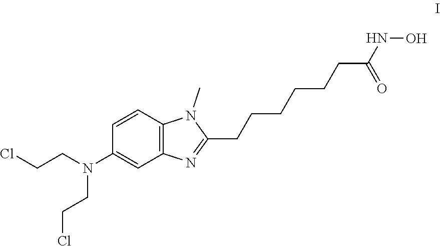 [SMILES: Cn1c(CCCCCCC(=O)NO)nc2cc(N(CCCl)CCCl)ccc21]

Biological assays showed that the compound of formula I potently inhibits HDAC enzyme (HDAC1 IC₅₀ of 9 nM).

There is a need for more effective treatments of Hodgkin lymphoma as many of the existing chemotherapy treatments are less than satisfactory.

### SUMMARY OF THE INVENTION

In a first aspect of the present invention there is provided a method of treating Hodgkin lymphoma in a patient in need thereof comprising administering to said patient an effective amount of a compound of formula I or a pharmacologically acceptable salt thereof:

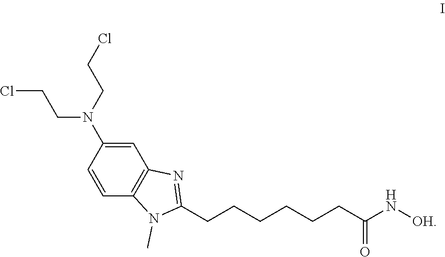 [SMILES: Cn1c(CCCCCCC(=O)NO)nc2cc(N(CCCl)CCCl)ccc21]

It has surprisingly been discovered that the compound of formula I or a pharmaceutically acceptable salt thereof is particularly effective in the treatment of Hodgkin lymphoma, with activity data showing strong sensitivity of all tested Hodgkin lymphoma cell lines to treatment with this compound. Thus, the need for a new and effective treatment of Hodgkin lymphoma is met by the present invention.

In a second aspect there is provided a compound of formula I or a pharmacologically acceptable salt thereof for use in a method of treating Hodgkin lymphoma in a patient in need thereof comprising administering to said patient an effective amount of said compound of formula I or a pharmacologically acceptable salt thereof.

In a third aspect of the present invention there is provided a combination comprising Brentuximab Vedotin and a compound of formula I or a pharmaceutically acceptable salt thereof.

In a fourth aspect of the present invention there is provided a kit comprising a combination according to the third aspect of the present invention, and optionally, instructions for treating a patient.

In a fifth aspect of the present invention there is provided a combination according to the third aspect of the present invention for use in therapy.

In a sixth aspect of the present invention there is provided a combination according to the third aspect of the present invention for use in a method of treating Hodgkin lymphoma in a patient in need thereof.

In a seventh aspect of the present invention there is provided a method of treating Hodgkin lymphoma in a patient in need thereof comprising administering to said patient a combination according to the third aspect of the present invention or a kit according to the fourth aspect of the invention.

### DESCRIPTION OF THE DRAWINGS

FIG. 1 is a plot of number of clones of four different Hodgkin lymphoma cell lines observed after culturing with the compound of formula I at six different concentrations;

FIG. 2 is a plot of the mitotic index for six different Hodgkin lymphoma cell lines observed after culturing with the compound of formula I;

FIGS. 3A to 3I are plots of cell proliferation for nine different Hodgkin lymphoma cell lines against concentration of the compound of formula I administered after culturing said cell line with said compound for 24 hours and 48 hours;

FIGS. 4A to 4E are plots of % survival of five different cell lines 0, 24, 48, 72 and 96 hours after administration of varying concentrations of the compound of formula I to said cell lines;

FIG. 5 shows the IC₅₀ values for bendamustine, the compound of formula I and vorinostat against five different Hodgkin lymphoma cell lines;

FIG. 6A is a plot of the number of cells for the bendamustine resistant cell line R100 against different concentrations of the compound of formula I measured 24, 48 and 72 hours after treatment with said compound;

FIG. 6B shows the IC₅₀ values for bendamustine, the compound of formula I and vorinostat against three different Hodgkin lymphoma cell lines;

FIG. 7 shows a plot of survival of fraction of surviving mice against time after injection with varying concentrations of Hodgkin lymphoma L428s cells;

FIGS. 8A and 8B show the two different dosage regimens used to test the NSG mice after xenografting with L428s Hodgkin lymphoma cells;

FIG. 9A is a plot of weight of mice against treatment time before and after treatment with the compound of formula I, vehicle and PBS; FIG. 9B is a plot of weight of mice against treatment time with the 60 mg/kg and 80 mg/kg dose;

FIG. 10A shows the incidence of lesions of different degrees of severity and the % of mice in which they occurred for each test group of mice treated with one 60 mg/kg dose EDO-S101, two 60 mg/kg doses of the compound of formula I separated by 3 weeks, an 80 mg/kg dose of the compound of formula I, vehicle and PBS as measured using immunohistochemistry;

FIG. 10B shows the incidence of lesions of different degrees of severity and the % of mice in which they occurred for each test group of mice treated with one 60 mg/kg dose of the compound of formula I, two 60 mg/kg doses of the compound of formula I separated by 3 weeks, an 80 mg/kg dose of the compound of formula I, vehicle and PBS as measured using immunofluorescence CD30 staining;

FIG. 11 shows a plot of the frequency of CD14 cells in mice after treatment with PBS, vehicle, one dose of 60 mg/kg of the compound of formula I and one dose of 80 mg/kg of the compound of formula I (plot A) and a plot of the frequency of CD14 cells in mice after treatment with PBS, vehicle and two doses of 60 mg/kg of the compound of formula I;

FIG. 12A shows a plot of % survival of L1236 bendamustine-naive cells against time for EDO-S101 alone, Brentuximab Vedotin alone, and the two in combination; and

FIG. 12B shows a plot of % survival of R100 bendamustine-resistant cells against time for EDO-S101 alone, Brentuximab Vedotin alone, and the two in combination.

### DETAILED DESCRIPTION OF THE INVENTION

In the present application, a number of general terms and phrases are used, which should be interpreted as follows.

“Patient” includes humans, non-human mammals (e.g., dogs, cats, rabbits, cattle, horses, sheep, goats, swine, deer, and the like) and non-mammals (e.g., birds, and the like).

“Pharmaceutically acceptable salts” means salts of compounds of the present invention which are pharmaceutically acceptable, as defined above, and which possess the desired pharmacological activity. Such salts include acid addition salts formed with inorganic acids, or with organic acids. Pharmaceutically acceptable salts also include base addition salts which may be formed when acidic protons present are capable of reacting with inorganic or organic bases. Generally, such salts are, for example, prepared by reacting the free acid or base forms of these compounds with a stoichiometric amount of the appropriate base or acid in water or in an organic solvent or in a mixture of the two. Generally, non-aqueous media like ether, ethyl acetate, ethanol, isopropanol or acetonitrile are preferred. Examples of the acid addition salts include mineral acid addition salts such as, for example, hydrochloride, hydrobromide, hydroiodide, sulfate, bisulfate, sulfamate, nitrate, phosphate, and organic acid addition salts such as, for example, acetate, trifluoroacetate, maleate, fumarate, citrate, oxalate, succinate, tartrate, salicylate, tosylate, lactate, naphthalenesulphonae, malate, mandelate, methanesulfonate and p-toluenesulfonate. Examples of the alkali addition salts include inorganic salts such as, for example, sodium, potassium, calcium and ammonium salts, and organic alkali salts such as, for example, ethylenediamine, ethanolamine, N,N-dialkylenethanolamine, triethanolamine and basic aminoacids salts.

“Hodgkin lymphoma” is a cancer of the lymphatic system and it is characterized by particular appearance under the microscope. These cells are called Reed Sternberg cells. In Hodgkin lymphoma, it is cells in the lymph nodes that have become cancerous. Hodgkin lymphoma is divided into two main classifications: classical Hodgkin lymphoma, which accounts for 90 to 95 percent of cases, and lymphocyte predominant Hodgkin lymphoma.

The compound of formula I or a pharmaceutically acceptable salt thereof may be used in the treatment of Hodgkin lymphoma, either alone or in combination with Brentuximab Vedotin.

In one aspect of the first, second, sixth and seventh aspects of the present invention, the methods of the present invention may be used in the treatment of relapsed/refractory Hodgkin lymphoma. It is known from the art that bendamustine is a very active agent in the treatment of relapsed-refractory Hodgkin lymphoma (see, for example, Moskowitz et al, J. Clin. Oncol. 2013, 31, 456-60), with a reported complete response rate of 29 to 35% and an overall response rate of 50 to 58%. As previously explained, the compound of formula I or a pharmaceutically acceptable salt thereof includes a bendamustine alkylating moiety as part of its molecular structure. However, it is known that about 40% of relapsed/refractory Hodgkin lymphoma patients are resistant to bendamustine. Evidence included herein shows that the activity shown against relapsed/refractory Hodgkin lymphoma by known alkylating agents such as bendamustine is also demonstrated by the compound of formula I or a pharmaceutically acceptable salt thereof, either alone or in combination with Brentuximab Vedotin while the resistance that is shown to bendamustine is not experienced with the compound of formula I. The compound of formula I or a pharmaceutically acceptable salt thereof, alone or in combination with Brentuximab Vedotin is consequently a very promising therapy for the treatment of relapsed/refractory Hodgkin lymphoma.

In the method of the first aspect and second aspect of the present invention, the compound of formula I or a pharmaceutically acceptable salt thereof is not administered in combination with a compound selected from proteasome inhibitors, glucocorticoids and class III receptor tyrosine kinase inhibitors. In another embodiment of the first and second aspect of the present invention, the method of treating Hodgkin lymphoma is a monotheraputic treatment consisting of the administration of the compound of formula I or a pharmacologically acceptable salt thereof to the patient in need thereof.

In one preferred aspect of the third, fourth, fifth, sixth and seventh aspects of the present invention, the composition, kit or method is synergistic.

In the present invention, the pharmaceutically acceptable salt of the compound of formula I may preferably be the hydrochloride, hydrobromide, hydroiodide, sulfate, bisulfate, sulfamate, nitrate, phosphate, citrate, methanesulfonate, trifluoroacetate, glutamate, glucuronate, glutarate, malate, maleate, oxalate, succinate, fumarate, tartrate, tosylate, mandelate, salicylate, lactate, p-toluenesulfonate, naphthalenesulfonate or acetate, and more preferably the acetate.

In the method of the first, second, sixth and seventh aspects of the present invention, the Hodgkin lymphoma is either classical Hodgkin lymphoma or lymphocyte predominant Hodgkin lymphoma.

The methods of the present invention are preferably directed to the treatment of classical Hodgkin lymphoma including nodular sclerosis classical Hodgkin lymphoma, mixed cellularity classical Hodgkin lymphoma, lymphocyte-depletion classical Hodgkin lymphoma and lymphocyte-rich classical Hodgkin lymphoma, and preferably nodular sclerosis classical Hodgkin lymphoma.

The methods of the present invention are alternatively directed to the treatment of lymphocyte predominant Hodgkin lymphoma such as nodular lymphocyte predominant Hodgkin lymphoma.

The therapeutically effective amount of the compound of formula I or a pharmacologically acceptable salt administered to the patient according to the methods of the first, second, sixth and seventh aspects of the present invention is an amount which confers a therapeutic effect in accordance with the present invention on the treated subject, at a reasonable benefit/risk ratio applicable to any medical treatment. The therapeutic effect may be objective (i.e. measurable by some test or marker) or subjective (i.e. subject gives an indication of or feels an effect). An effective amount of the compound of formula I or a pharmacologically acceptable salt thereof according to the first and second aspects of the present invention is believed to be one wherein the compound of formula I or a pharmacologically acceptable salt thereof is included at a dosage range of from 0.3 mg/m² to 300 mg/m² body surface area of the patient, preferably 1.5 mg/m² to 250 mg/m² body surface area of the patient, more preferably from 20 mg/m² to 150 mg/m² body surface area of the patient and most preferably from 40 mg/m² to 100 mg/m² body surface area of the patient, e.g. 40, 50, 60, 70, 80, 90 or 100 mg/m² body surface area of the patient.

In the combination according to the third aspect of the invention, the kit according to the fourth aspect of the present invention and the methods according to the fifth, sixth and seventh aspects of the present invention the compound of formula I or a pharmacologically acceptable salt thereof is included at a dosage range of from 0.3 mg/m² to 300 mg/m² body surface area of the patient, preferably 1.5 mg/m² to 150 mg/m² body surface area of the patient, more preferably from 10 mg/m² to 100 mg/m² body surface area of the patient and most preferably from 20 mg/m² to 80 mg/m² body surface area of the patient, e.g. 20, 30, 40, 50, 60, 70 or 80 mg/m² body surface area of the patient.

In the combination according to the third aspect of the invention, the kit according to the fourth aspect of the present invention and the methods according to the fifth, sixth and seventh aspects of the invention, BrentuximabVedotin is included at a dosage range of from 0.3 mg/m² to 300 mg/m² body surface area of the patient, more preferably from 0.5 mg/m² to 150 mg/m² body surface area of the patient and most preferably from 5 mg/m² to 100 mg/m² body surface area of the patient, e.g. 10, 20, 30, 40, 50, 60, 70, 80, 90 or 100 mg/m² body surface area of the patient.

The specific therapeutically effective dose level for any particular patient will depend upon a variety of factors including the specific form of Hodgkin lymphoma being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or contemporaneously with the specific compound employed; and like factors well known in the medical arts.

Suitable examples of the administration form of the compound of formula I or a pharmacologically acceptable salt thereof and medicament comprising the same according, either alone or in combination with Brentuximab Vedotin to the present invention include without limitation oral, topical, parenteral, sublingual, rectal, vaginal, ocular, and intranasal. Parenteral administration includes subcutaneous injections, intravenous, intramuscular, intrasternal injection or infusion techniques. Preferably, the compound of formula I or a pharmacologically acceptable salt thereof and medicament comprising the same, either alone or in combination with Brentuximab Vedotin are administered parenterally, and most preferably intravenously.

Preferably, the compound of formula I or a pharmacologically acceptable salt thereof is administered intravenously in monotherapy in accordance with the methods of the first and second aspects of the present invention to the patient in need thereof at a dosage level to the patient in need thereof of from 0.3 to 100 mg/m² body surface area of the patient, preferably intravenously to the patient in need thereof at a dosage level to the patient in need thereof of from 1.5 mg/m² to 250 mg/m² body surface area of the patient, more preferably intravenously to the patient in need thereof at a dosage level to the patient in need thereof of from 20 mg/m² to 150 mg/m² body surface area of the patient and most preferably intravenously to the patient in need thereof at a dosage level to the patient in need thereof of from 40 mg/m² to 100 mg/m²body surface area of the patient, e.g. 40, 50, 60, 70, 80, 90 or 100 mg/m² body surface area of the patient.

Preferably, the compound of formula I or a pharmacologically acceptable salt thereof is administered in combination therapy according to the fifth, sixth and seventh aspects of the present invention intravenously to the patient in need thereof at a dosage level to the patient in need thereof of from 0.3 mg/m² to 300 mg/m² body surface area of the patient, preferably intravenously to the patient in need thereof at a dosage level to the patient in need thereof of from 1.5 mg/m² to 150 mg/m² body surface area of the patient and most preferably intravenously to the patient in need thereof at a dosage level to the patient in need thereof of from 20 mg/m² to 80 mg/m² body surface area of the patient, e.g. 20, 30, 40, 50, 60, 70 or 80 mg/m² body surface area of the patient.

Preferably, BrentuximabVedotin administered in combination therapy according to the fifth, sixth and seventh aspects of the present invention intravenously to the patient in need thereof at a dosage level to the patient in need thereof of from 0.3 mg/m² to 300 mg/m² body surface area of the patient, more preferably from 0.5 mg/m² to 150 mg/m² body surface area of the patient and most preferably from 5 mg/m² to 100 mg/m² body surface area of the patient, e.g. 10, 20, 30, 40, 50, 60, 70, 80, 90 or 100 mg/m² body surface area of the patient.

In the first and second aspects of the present invention, the compound of formula I or a pharmacologically acceptable salt thereof or medicament comprising said compound is preferably administered to a patient in need thereof on days 1, 8 and 15 of a treatment cycle, i.e. one treatment per week. This cycle lasts for 4 to 6 weeks, depending upon the patient, followed by a break of 1 to 3 weeks. In an alternative treatment cycle, the compound of formula I or a pharmacologically acceptable salt thereof or medicament comprising said compound is administered to a patient in need thereof on days 1 and 22 of the treatment cycle, i.e. one treatment every third week. There are altogether 3 to 12 cycles of treatment, preferably 5-8, and most preferably 6, depending upon the patient. In another alternative treatment cycle, the compound of formula I or a pharmacologically acceptable salt thereof or medicament comprising said compound is administered to a patient in need thereof on days 1 and 8 of the treatment cycle. Thus, one cycle lasts for one week and is repeated for 4 cycles. There is a fourth alternative treatment cycle, in which the compound of formula I or a pharmacologically acceptable salt thereof or medicament comprising said compound is administered to a patient in need thereof on days 1 and 15 of the treatment cycle. Each cycle last for two weeks and is repeated for 4 cycles.

In the methods of the fifth, sixth and seventh aspects of the present invention, the compound of formula I or a pharmaceutically acceptable salt thereof and Brentuximab Vedotin are also preferably administered according to the same treatment cycles as in the method of the first and second aspects of the present invention. However, within these cycles, the compound of formula I or a pharmaceutically acceptable salt thereof and Brentuximab Vedotin may be administered concurrently, sequentially or separately. If they are administered separately they are preferably administered from 1 to 6 hours apart, preferably 2 to 3 hours apart.

In one preferred embodiment of the first and second aspects of the invention, the compound of formula I or a pharmacologically acceptable salt thereof is administered intravenously to the patient in need thereof at a dosage level of from 40 mg/m² to 100 mg/m² body surface area of the patient on days 1, 8 and 15 of a treatment cycle, for 4 to 6 weeks, followed by a break of 1 to 3 weeks.

In an alternative embodiment of the first and second aspects of the invention, the compound of formula I or a pharmacologically acceptable salt thereof is administered intravenously to the patient in need thereof at a dosage level of from 40 mg/m² to 100 mg/m² body surface area of the patient on days 1 and 22 of a treatment cycle, for 3-12 consecutive cycles, more preferably 5-8 cycles and most preferably 6 cycles.

In one preferred embodiment of the first and second aspects of the invention, the compound of formula I or a pharmacologically acceptable salt thereof is administered intravenously to the patient in need thereof at a dosage level of 40 mg/m² to 100 mg/m² body surface area of the patient on days 1 and 15 of a treatment cycle, for 4 consecutive cycles.

In another preferred embodiment of the first and second aspects of the invention, the compound of formula I or a pharmacologically acceptable salt thereof is administered intravenously to the patient in need thereof at a dosage level of 40 mg/m² to 100 mg/m² body surface area of the patient on days 1 and 8 of a treatment cycle, for 4 consecutive cycles.

In one preferred embodiment of the fifth, sixth and seventh aspects of the present invention, the molar ratio of the compound of formula I or a pharmaceutically acceptable salt thereof to Brentuximab Vedotin is from 1:0.1 to 1:5, preferably 1:0.25 to 1:2.

In one preferred embodiment of the fifth, sixth and seventh aspects of the present invention, the compound of formula I or a pharmacologically acceptable salt thereof and Brentuximab Vedotin are administered separately over a period of 2 to 3 hours intravenously to the patient in need thereof on days 1, 8 and 15 of a treatment cycle, for 4 to 6 weeks, followed by a break of 1 to 3 weeks.

In an alternative embodiment of the fifth, sixth and seventh aspects of the present invention, the compound of formula I or a pharmacologically acceptable salt thereof and Brentuximab Vedotin are administered separately over a period of 2 to 3 hours intravenously to the patient in need thereof on days 1 and 22 of the treatment cycle, for 3 to 12 cycles of treatment, preferably 5 to 8 cycles, and most preferably 6 cycles.

In a further alternative embodiment of the fifth, sixth and seventh aspects of the present invention, the compound of formula I or a pharmacologically acceptable salt thereof and Brentuximab Vedotin are administered separately over a period of 2 to 3 hours intravenously to the patient in need thereof on days 1 and 8 of the treatment cycle, and this is repeated for 4 cycles.

In another embodiment of the fifth, sixth and seventh aspects of the present invention, the compound of formula I or a pharmacologically acceptable salt thereof and Brentuximab Vedotin are administered separately over a period of 2 to 3 hours intravenously to the patient in need thereof on days 1 and 15 of the treatment cycle, and this is repeated for 4 cycles.

In one preferred embodiment of the fifth, sixth and seventh aspects of the present invention, the compound of the compound of formula I or a pharmacologically acceptable salt thereof and Brentuximab Vedotin are administered intravenously to the patient in need thereof, wherein the compound of formula I or a pharmacologically acceptable salt thereof is administered intravenously at a dosage level of from 20 mg/m² to 80 mg/m² body surface area of the patient and Brentuximab Vedotin is administered intravenously to the patient in need thereof at a dosage level of from 5 mg/m² to 100 mg/m² body surface area of the patient on days 1, 8 and 15 of a treatment cycle, wherein the compound of formula I or a pharmaceutically acceptable salt thereof and Brentuximab Vedotin are administered separately over a period of 2 to 3 hours, and wherein the administration cycle is performed over a period of 4 to 6 weeks, followed by a break of 1 to 3 weeks.

In an alternative embodiment the fifth, sixth and seventh aspects of the present invention, the compound of the compound of formula I or a pharmacologically acceptable salt thereof and Brentuximab Vedotin are administered intravenously to the patient in need thereof, wherein the compound of formula I or a pharmacologically acceptable salt thereof is administered intravenously at a dosage level of from 20 mg/m² to 80 mg/m² body surface area of the patient and Brentuximab Vedotin is administered intravenously to the patient in need thereof at a dosage level of from 5 mg/m² to 100 mg/m² body surface area of the patient on days 1 and 22 of a treatment cycle, wherein the compound of formula I or a pharmaceutically acceptable salt thereof and Brentuximab Vedotin are administered separately over a period of 2 to 3 hours, and wherein the administration cycle is performed over 3-12 consecutive cycles, more preferably 5-8 cycles and most preferably 6 cycles.

In another preferred embodiment of the fifth, sixth and seventh aspects of the present invention, the compound of formula I or a pharmacologically acceptable salt thereof and Brentuximab Vedotin are administered intravenously to the patient in need thereof, wherein the compound of formula I or a pharmacologically acceptable salt thereof is administered intravenously at a dosage level of from 20 mg/m² to 80 mg/m² body surface area of the patient and Brentuximab Vedotin is administered intravenously to the patient in need thereof at a dosage level of from 5 mg/m² to 100 mg/m² body surface area of the patient, wherein the compound of formula I or a pharmaceutically acceptable salt thereof and Brentuximab Vedotin are administered separately over a period of 2 to 3 hours, and wherein the administration cycle is performed on days 1 and 15 of a treatment cycle, for 4 consecutive cycles.

In another preferred embodiment of the fifth, sixth and seventh aspects of the present invention, the compound of formula I or a pharmacologically acceptable salt thereof and Brentuximab Vedotin are administered intravenously to the patient in need thereof, wherein the compound of formula I or a pharmacologically acceptable salt thereof is administered intravenously at a dosage level of from 20 mg/m² to 80 mg/m² body surface area of the patient and Brentuximab Vedotin is administered intravenously to the patient in need thereof at a dosage level of from 5 mg/m² to 100 mg/m² body surface area of the patient, wherein the compound of formula I or a pharmaceutically acceptable salt thereof and Brentuximab Vedotin are administered separately over a period of 2 to 3 hours, and wherein the administration cycle is performed on days 1 and 8 of a treatment cycle, for 4 consecutive cycles.

In the fourth aspect of the present invention, there is provided a kit comprising a combination according to the second aspect of the present invention and, optionally, instructions for treating a patient. Typically, a kit can comprise a compound of formula I or pharmaceutically acceptable salt thereof and Brentuximab Vedotin together with instructions for treating a patient. Each active agent can be provided in a suitable container. The kit may further comprise a delivery system, e.g. for the compound of formula I or pharmaceutically acceptable salt thereof and Brentuximab Vedotin.

The instructions may advise administering BrentuximabVedotin and the compound of formula I or a pharmaceutically acceptable salt thereof concurrently, sequentially or separately according to variables such as the state of the Hodgkin lymphoma being treated; the activity of the specific compounds employed; the age, body weight, general health, sex and diet of the patient; the time of administration, route of administration, and rate of excretion of the specific compounds employed; the duration of the treatment; drugs used in combination or contemporaneously with the specific compounds employed; and like factors well known in the medical arts. Preferred kits according to the fourth aspect of the present invention include those comprising the preferred combinations of the present invention as described and exemplified above.

In a further embodiment of the methods of the first, second, fifth, sixth and seventh aspects of the present invention, the patient in need of said treatment is given radiotherapy prior to or after treatment of the Hodgkin lymphoma with the compound of formula I or a pharmacologically acceptable salt thereof (and, in the fourth aspect the Brentuximab Vedotin). Preferably, the patient is given radiotherapy treatment prior to the treatment with the compound of formula I or a pharmacologically acceptable salt thereof (and, in the fourth aspect the Brentuximab Vedotin) or the medicament comprising the same. The radiotherapy may be given at a dose of 1 to 5 Gy over 5 consecutive days and preferably 2 Gy over 5 consecutive days.

When intended for oral administration, the compound of formula I or a pharmacologically acceptable salt thereof or medicament comprising the same for use in the methods of treating Hodgkin lymphoma of the present invention or the combination of the third aspect of the present invention or in the kit according to the fourth aspect of the present invention may be in solid or liquid form, where semi-solid, semi-liquid, suspension and gel forms are included within the forms considered herein as either solid or liquid.

The compound of formula I or a pharmacologically acceptable salt thereof or medicament comprising the same for use in the methods of treating Hodgkin lymphoma of the present invention or the combination of the third aspect of the present invention or in the kit according to the fourth aspect of the present invention can be prepared for administration using methodology well known in the pharmaceutical art. Examples of suitable pharmaceutical formulations and carriers are described in “Remington's Pharmaceutical Sciences” by E. W. Martin.

As a solid composition for oral administration, the compound of formula I or a pharmacologically acceptable salt thereof or medicament comprising the same for use in the methods of treating Hodgkin lymphoma of the present invention or the combination of the second aspect of the present invention or in the kit according to the third aspect of the present invention can be formulated into a powder, granule, compressed tablet, pill, capsule, chewing gum, wafer or the like form. Such a solid composition typically contains one or more inert diluents, either as a single tablet comprising all active agents or as a number of separate solid compositions, each comprising a single active agent of the combination of the present invention (in the case of the kit). In addition, one or more of the following can be present: binders such as carboxymethylcellulose, ethyl cellulose, microcrystalline cellulose, or gelatin; excipients such as starch, lactose or dextrins, disintegrating agents such as alginic acid, sodium alginate, corn starch and the like; lubricants such as magnesium stearate; glidants such as colloidal silicon dioxide; sweetening agents such as sucrose or saccharin; a flavoring agent such as peppermint, methyl salicylate or orange flavoring; and a coloring agent.

When the compound of formula I or a pharmacologically acceptable salt thereof or medicament comprising the same for use in the methods of treating Hodgkin lymphoma of the present invention or the combination of the third aspect of the present invention or in the kit according to the fourth aspect of the present invention is in the form of a capsule (e. g. a gelatin capsule), it can contain, in addition to materials of the above type, a liquid carrier such as polyethylene glycol, cyclodextrin or a fatty oil.

The compound of formula I or a pharmacologically acceptable salt thereof or medicament comprising the same for use in the methods of treating Hodgkin lymphoma of the present invention or the combination of the third aspect of the present invention or in the kit according to the fourth aspect of the present invention can be in the form of a liquid, e. g. an elixir, syrup, solution, emulsion or suspension. The liquid can be useful for oral administration or for delivery by injection. When intended for oral administration, a compound of formula I or a pharmacologically acceptable salt thereof or medicament comprising the same for use in the methods of treating Hodgkin lymphoma of the present invention or the combination of the third aspect of the present invention or in the kit according to the fourth aspect of the present invention can comprise one or more of a sweetening agent, preservatives, dye/colorant and flavor enhancer. In a compound of formula I or a pharmacologically acceptable salt thereof or medicament comprising the same for use in the method of treating Hodgkin lymphoma of the present invention for administration by injection, one or more of a surfactant, preservative, wetting agent, dispersing agent, suspending agent, buffer, stabilizer and isotonic agent can also be included.

Brentuximab Vedotin (trade name Adcetris®) is formulated for delivery by injection. It is stored as a white concentrated powder which is reconstituted with a sodium chloride solution for delivery to the desired concentration.

The preferred route of administration is parenteral administration including, but not limited to, intradermal, intramuscular, intraperitoneal, intravenous, subcutaneous, intranasal, epidural, intranasal, intracerebral, intraventricular, intrathecal, intravaginal or transdermal. The preferred mode of administration is left to the discretion of the practitioner, and will depend in part upon the site of the medical condition (such as the site of cancer). In a more preferred embodiment, the compound of formula I or a pharmacologically acceptable salt thereof is administered intravenously.

The liquid compound of formula I or a pharmacologically acceptable salt thereof or medicament comprising the same for use in the methods of treating Hodgkin lymphoma of the present invention or in the combination of the third aspect of the present invention or in the kit according to the fourth aspect of the present invention, whether they are solutions, suspensions or other like form, can also include one or more of the following: sterile diluents such as water for injection, saline solution, preferably physiological saline, Ringer's solution, isotonic sodium chloride, fixed oils such as synthetic mono or digylcerides, polyethylene glycols, glycerin, or other solvents; antibacterial agents such as benzyl alcohol or methyl paraben; and agents for the adjustment of tonicity such as sodium chloride or dextrose. A parenteral combination or composition can be enclosed in an ampoule, a disposable syringe or a multiple-dose vial made of glass, plastic or other material. Physiological saline is a preferred adjuvant.

The compound of formula I or a pharmacologically acceptable salt thereof or medicament comprising the same for use in the method of treating Hodgkin lymphoma of the present invention can be administered by any convenient route, for example by infusion or bolus injection, by absorption through epithelial or mucocutaneous linings, and preferably by bolus injection.

### EXAMPLES

In the following examples, the compound having the following formula I is referred to as EDO-S101.

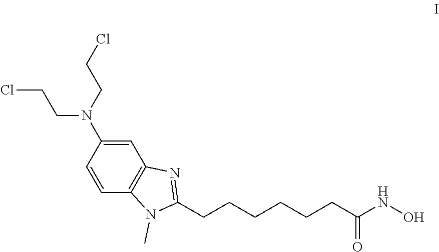 [SMILES: Cn1c(CCCCCCC(=O)NO)nc2cc(N(CCCl)CCCl)ccc21]

EDO-S101 may be prepared as described in Example 6 of WO-A-2010/085377.

### Example 1

### Hodgkin Lymphoma Cell Lines

In all examples, the human Hodgkin lymphoma-derived cell lines L428, L428-c, KMH2, L591, L540, L1236, HDLM2 and SUP-HD were obtained from Dr Steffen Junker (Aarhus University Denmark). All cell lines were cultured in Gibco RPMI 1640 medium supplemented with Glutamax, 10% FBS and antibiotics at 37° C. Some cell lines exhibited higher growth and others relatively slow growth. The immunophenotype, P53 status and MSI characterization of the Hodgkin lymphoma cell lines was determined using anti-CD30, CD15, CD20 and CD14 antibodies.

Table 1 below shows the origin and histological type of the cell lines. Five cell lines were nodular sclerosis Hodgkin lymphoma cell lines and two cell lines were mixed cellularity Hodgkin lymphoma cell lines, thus covering the two most common forms of Hodgkin lymphoma.

TABLE 1

| | | | | Derived patients | |
|---|---|---|---|---|---|
| Cell line | Origin | Histology Type | EBV status | (age) | |
| KMH2 | B-cell | mixed cellularity | − | Male | (37) |
| L1236 | B-cell | mixed cellularity | − | Male | (34) |
| L428 | B-cell | nodular sclerosis | − | Female | (37) |
| SUP-HD1 | B-cell | nodular sclerosis | − | Male | (33) |
| L591 | B-cell | nodular sclerosis | + | Female | (31) |
| HDLM2 | T-cell | nodular sclerosis | − | Male | (74) |
| L540 | T-cell | nodular sclerosis | − | Female | (20) |

EBV status=indication of presence or otherwise of the Epstein-Barr virus genome.

To determine the functional status of p53, a functional yeast assay was performed as described in Flaman et al., Proc. Natl. Acad. Sci. USA 1995 Apr. 25, 92 (9), 3963-3967. This analysis showed the showed presence of 100% yeast colonies of red color in three cell lines, indicating nonfunctional status of p53. Sequencing of p53 cDNA confirmed the presence of mutations in L428 (exon 4), L1236 (exon 10-11) and HDLM2 (exon 8-11) (see Table 2 below).

TABLE 2

| Cell line | P53 mutated | Microsatellite (MSI) |
|---|---|---|
| KMH2 | − | 3/5 |
| L1236 | + (exon10-11) | 0/5 |
| L428 | + (exon4) | 4/5 |
| SUP-HD1 | − | NA |
| L591 | − | 1/5 |
| HDLM2 | + (exon 8-11) | 3/5 |
| L540 | − | 1/5 |

Microsatellite instability (MSI) is the condition of genetic hypermutability that results from impaired DNA Mismatch Repair. In other words, microsatellite instability is the phenotypic evidence that Mismatch Repair is not functioning normally. DNA Mismatch Repair corrects errors that spontaneously occur during DNA replication like single base mismatches or short insertions and deletions. The proteins involved in Mismatch Repair form a complex that binds to the mismatch, identifies the correct strand of DNA, then subsequently excises the error and repairs the mismatch. Cells with abnormally functioning Mismatch Repair tend to accumulate errors rather than correcting those errors. As a result, gene sequences are not preserved faithfully through DNA replication, and novel microsatellite fragments are created. Microsatellite instability is detected by PCR based assays that reveal these novel microsatellites.

Microsatellites are repeated sequences of DNA. These sequences can be made of repeating units of 1-6 base pairs in length. Although the length of these microsatellites is highly variable from person to person (part of DNA “fingerprint”), each individual has microsatellites of a set length. The most common microsatellite in humans is a dinucleotide repeat of CA, which occurs tens of thousands of times across the genome. Microsatellites are also known as simple sequence repeats (SSRs).

### Example 2

### Clonogenic Survival of Hodgkin Lymphoma Cell Lines Treated by EDO-S101

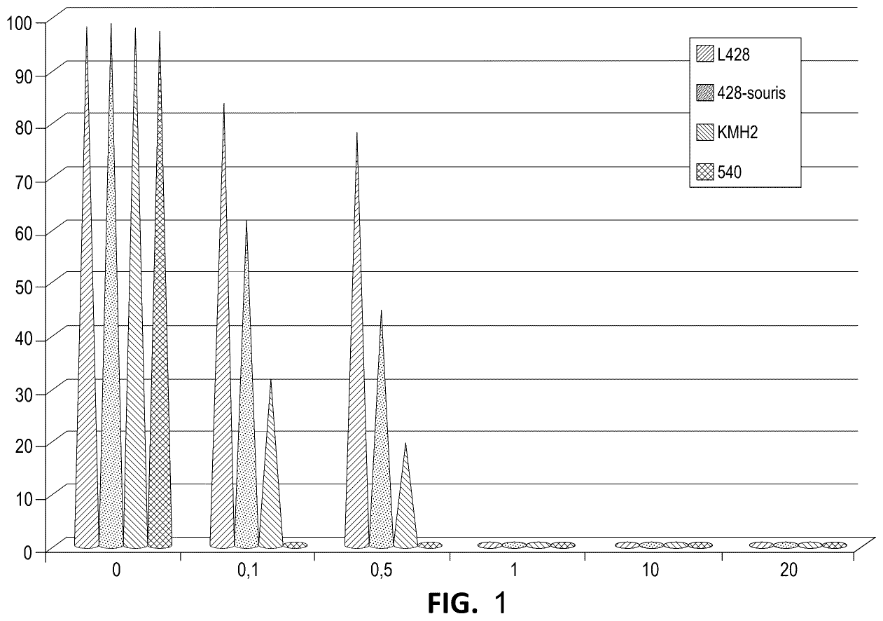

This test was performed on four Hodgkin lymphoma cell lines (L428, L428s, KMH2 and L540). Each was cultured with EDO-S101 at doses of 0, 0.1, 0.5, 1, 10 and 20 μmol in DMSO. The cell lines were cultured in methylcellulose (Stem cell Technologies) at a concentration of 10⁵/ml at 37° C. and 5% CO₂. The surviving fraction was determined by measuring the viability of colonies generated before and after exposure. Only colonies with more than 50 cells were counted. The results are shown in FIG. 1. Each data point represents the average and SD from three independent experiments performed in triplicate.

No clone was observed for the L540 cell line following treatment at any dose indicating a high sensitivity of this cell line to EDO-S101 treatment.

Similarly, the KMH2 cell line exhibited a high sensitivity, as only a few clones, characterized by their large size, were observed at the lowest doses of EDO-101 tested (0.1 and 0.5 μmol).

L428 and L428s also exhibited a high sensitivity, with only macrophagic clones being observed after exposure to very low doses (0.1 and 0.5 μmol).

It can further be seen that both p53 positive and negative cell lines were sensitive to EDO-S101 treatment. This is important as it is known from the art (e.g. see Wilson et al, Blood, 89, 2, 1997, 601-609) that the p53 gene plays a part in clinical drug resistance in Hodgkin lymphoma. The fact that EDO-S101 is active against both p53+and p53-Hodgkin cell lines is strongly suggestive that it may be active in the treatment of relapsed/refractory Hodgkin lymphoma.

### Example 3

### Mitotic Index and Induced Chromosomal Aberrations After EDO-S101 Treatment

The mitotic index and chromosomal aberrations for each of the cell lines were evaluated after treatment with either 20 μmol or 10 μmol EDO-S101 in DMSO. Each cell line was cultured at 37° C. in the presence of RPMI 1640 supplemented with 10% FCS and antibiotics. Colcemid (0.1 ug/ml) was added 2 h before harvesting and slides with chromosomes in metaphase were prepared following the standard methanol/acetic acid (3/1, v/v) procedure. The slides were stored at −20° C. until use.

FISH was performed using a combination of standard procedures from the recommended protocols for chromosome analysis, following telomere and centromere staining, using PNA probes. In all, 100 metaphases were scored per in vitro dose exposure.

No metaphases were observed following the 20 μmol EDO-S101 treatment for any cell line and only a few metaphases of poor quality were observed following 10 μmol EDO-S101 treatment. The metaphase of the KMH2 cell line exhibited numerous chromosomal aberrations (detected using telomere and centromere staining).

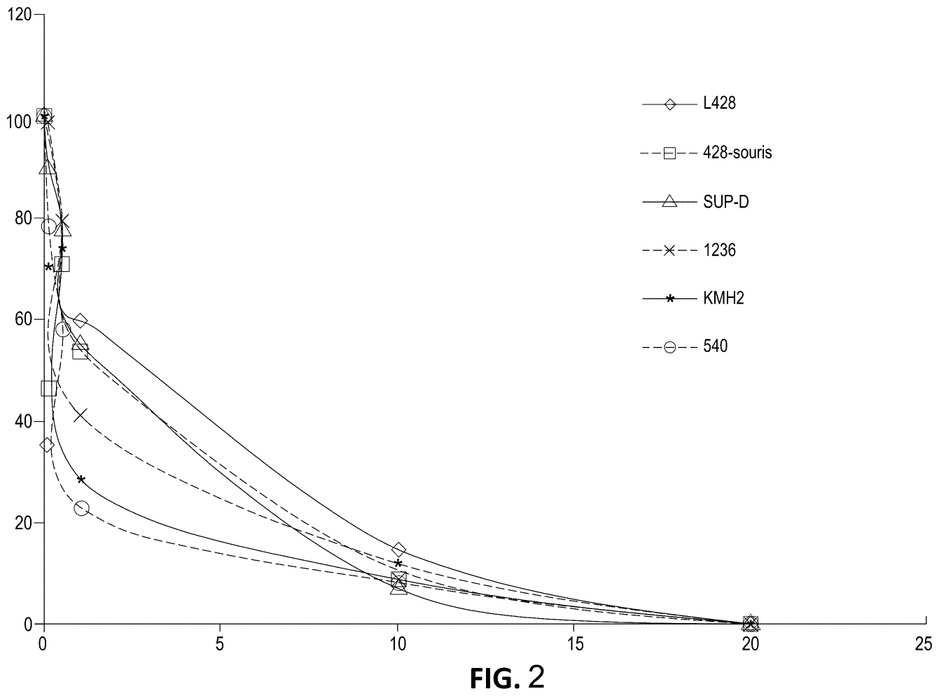

Of note, the L540 and KMH2 cell lines exhibited a very low mitotic index. The mitotic index assay, the results of which are shown in FIG. 2, demonstrated a remarkably high sensitivity of all Hodgkin lymphoma cell lines to EDO-S101 after only 24 h of treatment. The mitotic index was determined by measurement of the sum of all cells in prophase, metaphase, anaphase and telophase divided by the total number of cells.

### Example 4

### Proliferation and IC50 Determination

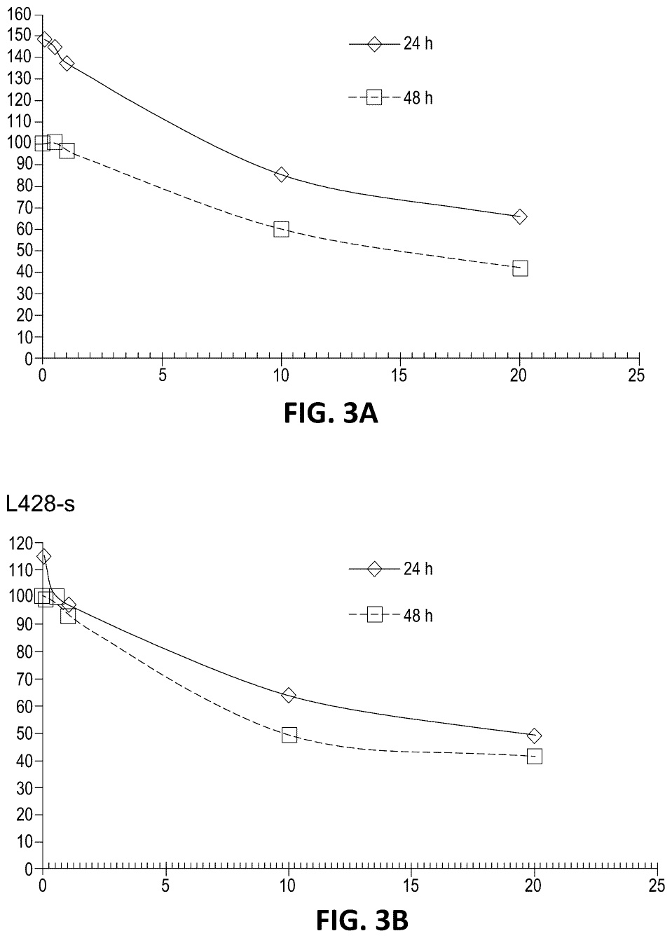

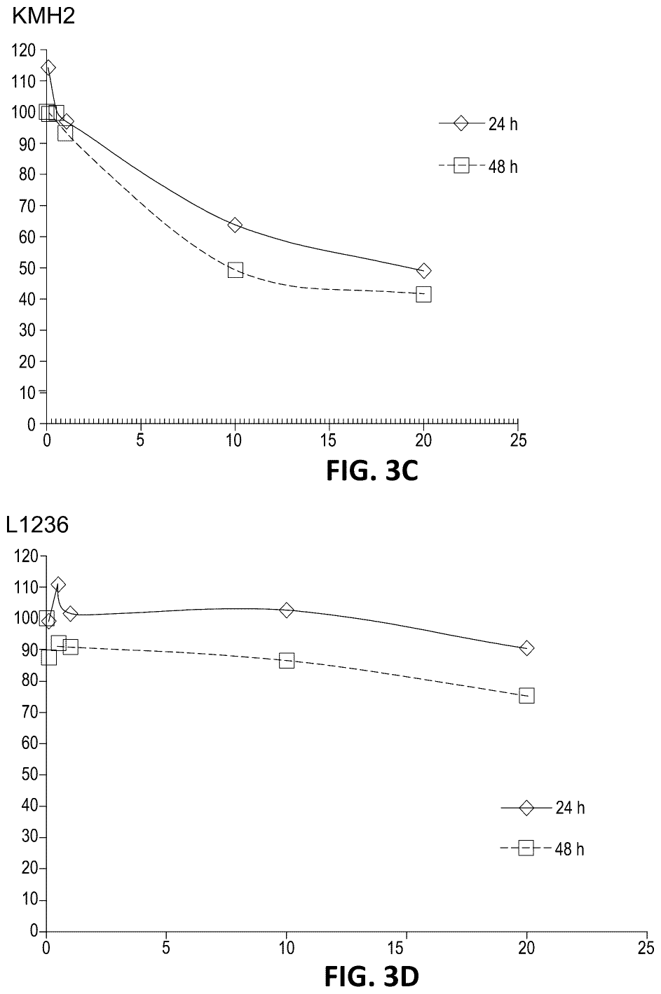

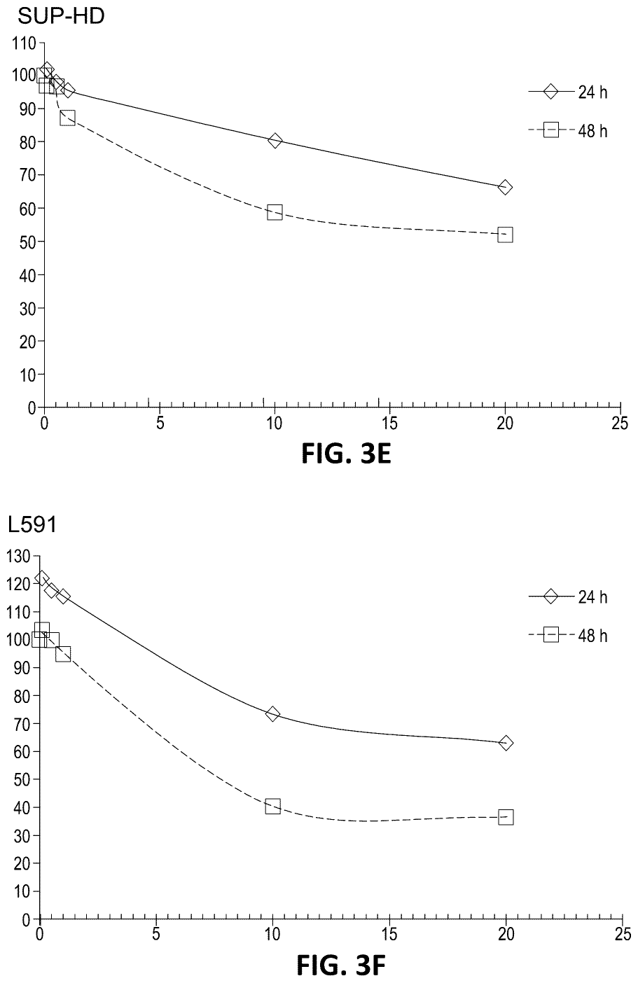

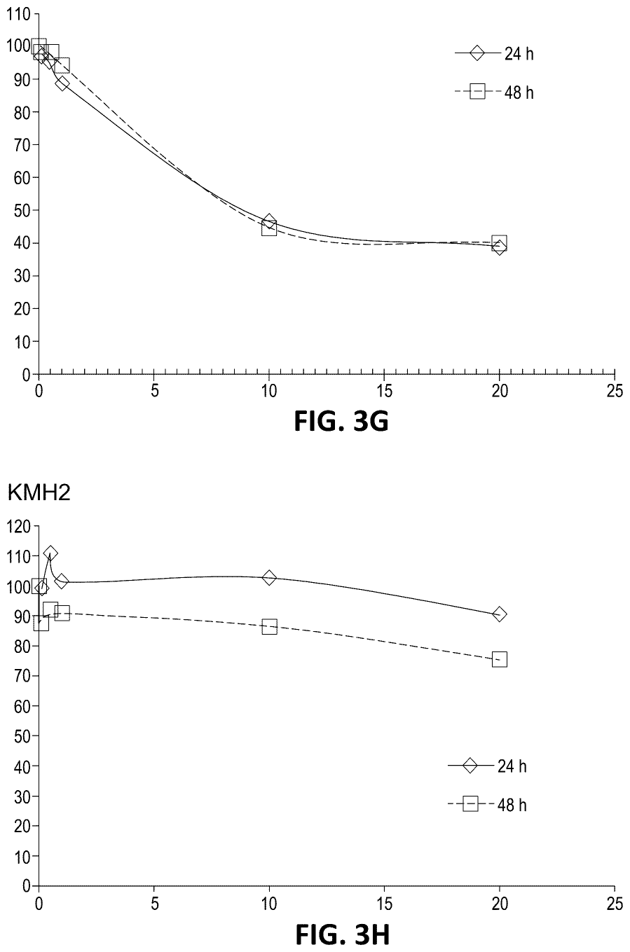

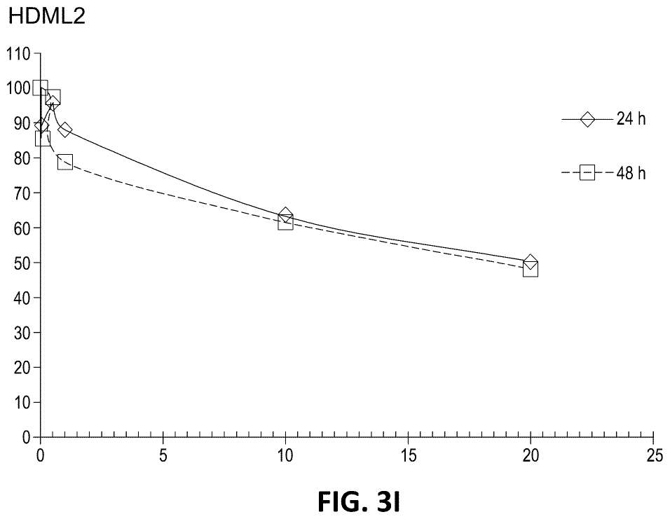

Nine Hodgkin lymphoma cell lines were cultured at 37° C. in 24-well plates at a concentration of 0.3×10⁶ cells/ml in the presence of EDO-S101 at concentrations of 0, 5, 10, 15 and 20 μmol in DMSO for 24 hours and 48 hours and the proliferation of the cells was determined using a non-radioactive cell proliferation MTS assay with CellTiter 96® Aqueous One solution reagent obtained from Promega® (this is a colorimetric method for determining the number of viable cells in a proliferation comprising a tetrazolium compound [3-(4,5-dimethylthiazol-2-yl)-5-(3-carboxymethoxyphenyl)-2-(4-sulfophenyl)-2H-tetrazolium, inner salt; MTS). The proliferation was plotted against concentration of EDO-S101 for treatment after both 24 and 48 hours. As can be seen from FIGS. 3A to 3I and Table 3 below, EDO-S101 showed very strong anti-proliferative activity against all the Hodgkin lymphoma cell lines, with the IC50 ranging between 6.3 μM and 1.6 μM at 48 h following manual counting using Trypan blue.

TABLE 3

| Cell Lines | Cell Lines |
|---|---|
| L428 | L428 |
| L428-s | L428-s |
| L540 | L540 |
| L1236 | L1236 |
| SUP-HD | SUP-HD |
| L591 | L591 |
| HDLM2 | HDLM2 |
| KMH2 | KMH2 |

There are some difference between the results obtained using this test compared to that obtained using manual scoring or clonogenic survival. This difference was due essentially to the sensitivity and the specificity of this test. This test measured the activity of the proliferation of the Hodgkin lymphoma cells and not the proliferation in terms of cell division.

### Example 5

### Dose and Time-Dependent Effects of EDO-S101 on Hodgkin Lymphoma Cell Growth

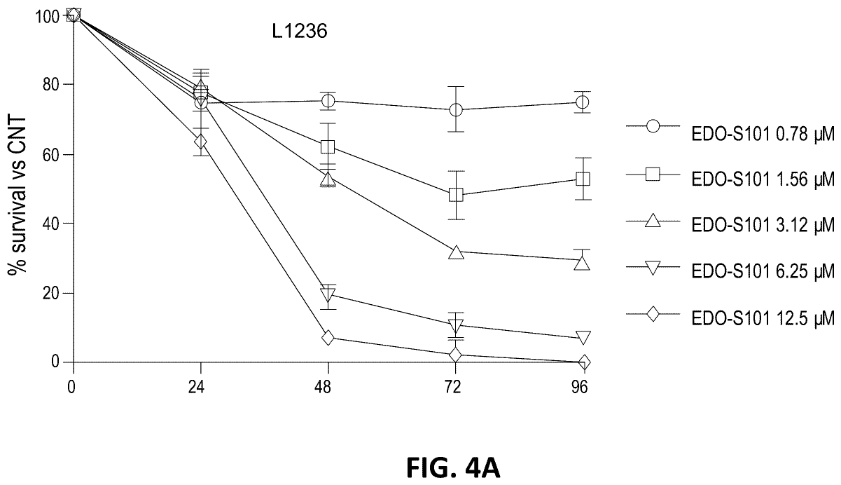

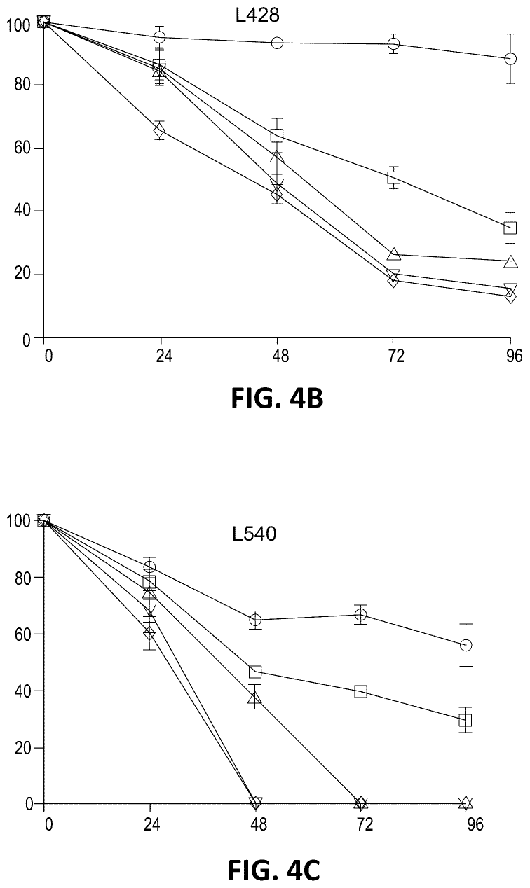

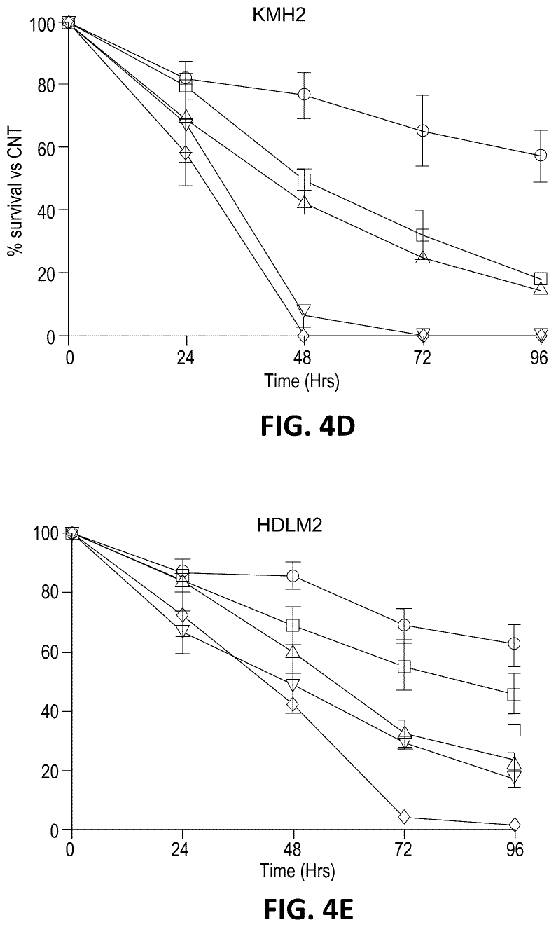

Five Hodgkin lymphoma cell lines (KMH2, HDML2, L540, L1236 and L426) were cultured in the presence of EDO-S101 dissolved in DMSO at concentrations of 0.78 μM, 1.56 μM, 3.12 μM, 6.25 μM and 12.5 μM at 37° C. Control cells were cultured in the same final concentration of DMSO. The cell viability, calculated as the % cell survival vs. control (CNT) was assessed at 24, 48, 72 and 96 hours through a Countess™ automated cell counter (Invitrogen), each experiment being repeated three times and the results given being the mean of the three results±SD. The results are shown in FIGS. 4A to 4E.

### Example 6

### Comparison of IC50s and Anti-proliferative Effects of Bendamustine, Vorinostat and EDO-S101 Against Hodgkin Lymphoma Cell Lines

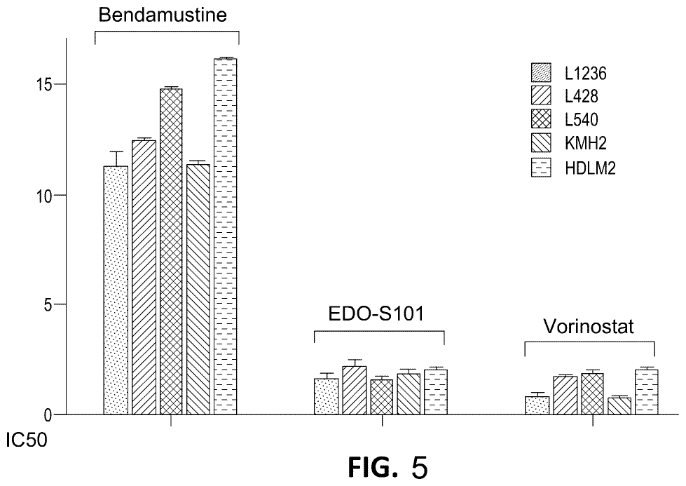

(a) Five Hodgkin lymphoma cell lines (KMH2, HDML2, L540, L1236 and L426) were cultured separately in the presence of bendamustine, vorinostat and EDO-S101 dissolved in DMSO at 37° C. L1236-derived Hodgkin lymphoma cell lines were selected as they have an acquired stable resistance to bendamustine (R100) or an ability to display continuous growth upon extended exposure to sub-lethal concentrations of bendamustine (R25, R50 and R75). A detailed description of the L1236-R Hodgkin lymphoma cell lines is disclosed in De Filippi et al, ASH 2015, Abstract. #2479. In these tests, the L1236-100R bendamustine resistant strain was used. As can be seen from FIG. 5, the IC50 values for all cell lines cultured with bendamustine were high, ranging between 11 and 17 μM. On the other hand, the IC50 values for all Hodgkin lymphoma cell lines tested, including the bendamustine resistant cell line L1236-R were of an order of magnitude lower for EDO-S101, in the range 1 to 2 μM and similar to the known anti-lymphoma agent vorinostat.

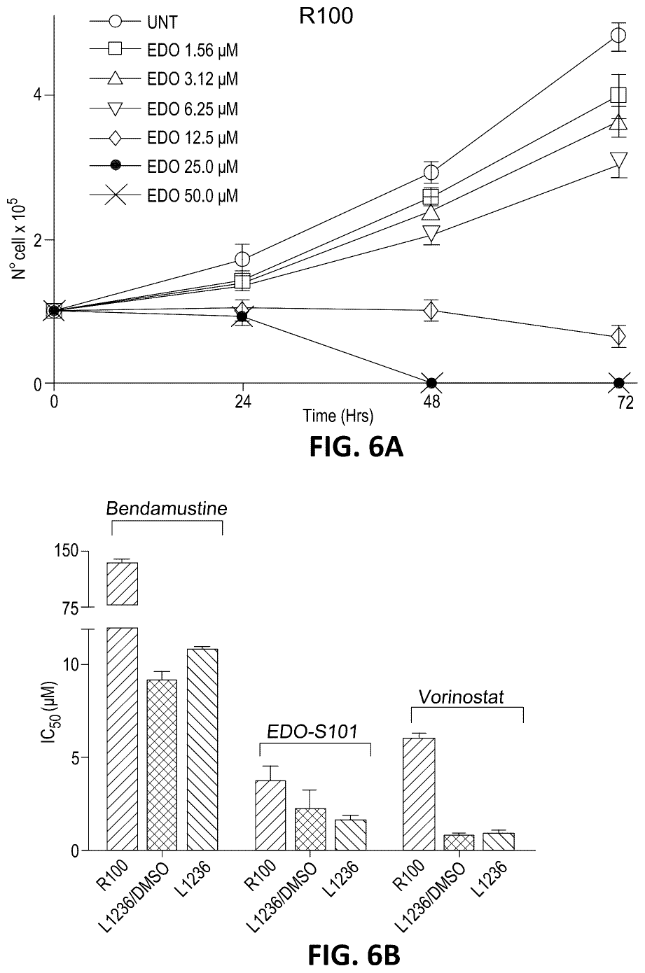

(b) The bendamustine-resistant Hodgkin lymphoma cell line L1236-R100, non-resistant L1236 Hodgkin lymphoma cell line cultured in DMSO L1236/DMSO and L1236 was cultured in the presence of EDO-S101 dissolved in DMSO at concentrations of 0.78 μM, 1.56 μM, 3.12 μM, 6.25 UM and 12.5 μM. Control cells were cultured in the same final concentration of DMSO. The number of viable cells was assessed at 24, 48 and 72 hours through a Countess™ automated cell counter (Invitrogen), each experiment being repeated three times and the results given being the mean of the three results±SD. These results were used to produce a plot of the number of viable cells against time at 24, 48 and 72 hours for each of the three cell lines after treating with bendamustine, EDO-S101 and vorinostat (see FIG. 6A for the R100 cell line) and the IC50 (μM) for each of bendamustine, EDO-S101 and vorinostat against each of the three Hodgkin lymphoma cell lines tested (se FIG. 6B).

As will be immediately apparent, and as would be expected, the IC50 values for bendamustine against the R100 cell line are very high indeed, in the region of 140 M, while it also shows high IC50 values against the non-resistant L1236 Hodgkin lymphoma cell lines. Thus, the anti-proliferative activity of bendamustine is quite low. Surprisingly, however, EDO-S101 showed remarkable anti-proliferative activity against all Hodgkin lymphoma cell lines tested, including the R100 cell line, with IC50 values in the range of approximately 1 to 3 μM, approximately two orders of magnitude greater than for bendamustine.

It can also be seen from FIG. 6A that at the higher concentrations of EDO-S101 the number of viable cells after exposure to the compound tails off rapidly, with no viable cells being observed 48 hours after treatment with 50 μM EDO-S101. It is also noteworthy that the anti-proliferative activity of EDO-S101 against the bendamustine-resistant Hodgkin lymphoma cell line R100 is greater than that of vorinostat. The evidence of this example provides strong proof of the anti-proliferative activity of EDO-S101 against Hodgkin lymphoma cell lines that have to date proven difficult to treat, making it a promising candidate for the treatment of Hodgkin lymphoma.

### Example 7

### Assessment of Apoptosis Induced by EDO-S101

This assay was performed on 7 cell lines (HDLM2 was excluded due to the low number of available cells) at 24 h and 48 h following EDO-S101 treatment (5 μM, DMSO). Control cells were cultured in the same final concentration of DMSO. Only viable cells (using Hoechst staining) were focused on, and all necrotic cells were excluded. Apoptosis was determined using annexin V-FITC and Hoechst (33342) double staining according to the manufacturer's instructions (BD Biosciences). Cell-cycle fractions were determined by Hoechst nuclear staining. Briefly, cells were harvested, washed in PBS, incubated with annexin V-FITC and Hoechst solution for 30 minutes at room temperature. Data were collected using a FACSCalibur flow cytometer (BD Biosciences) and analyzed with FlowJo Version 7.5.5 obtainable from FlowJo, LLC. Results represent the mean value of 3 independent experiments.

All cells exhibited high sensitivity to EDO-S101 for apoptosis reflecting the major DNA repair deficiency. L540 cell exhibited a higher sensitivity as well as all others cell lines independent of P53 status.

### Example 8

### Development of a Xenograft Model of Hodgkin Lymphoma

The human HL-derived cell lines L428, KMH2, L591, HDLM2, L540, SUP-HD and L1236 were cultured in Gibco RPMI 1640 medium supplemented with Glutamax and 10% FBS and 1% antibiotics at 37° C. The different cell lines exhibited varying division rates.

Immunodeficient NOD-SCID-gammac−/−(NSG) mice aged 5 to 6 weeks were purchased from Charles River (NOD.Cg-Prkdcscid II2rgtm1Wjl/SzJ). The mice arrived two weeks before the beginning of the experiments and were housed in micro-isolator cages during the entire course of the study. Immediately prior to the injection of Hodgkin lymphoma cells, mice were exposed to 3Gy gamma irradiation using an IBL637 ¹³⁷Cs irradiator at a dose rate of 0.61Gy/min. From 10³ to 10⁶ cells, either from the original cell lines or following amplification, were injected intravenously via the retro-orbital sinus. The mice were sacrificed by cervical dislocation and different organs were analysed and tumoural infiltration examined using multiple techniques.

The injected L428-s cell line served as a control for all experiments.

The first step of the in vivo study consisted of the injection of 10⁶ cells of L428-c, KMH2-c, L591 and L1236 per NSG mouse. Of note, while 100% of the mice injected with L428-c displayed tumour infiltration, this was true for only 66% of those injected with KMH2-c. There was a high level of infiltration of L428-c Hodgkin lymphoma cells into various organs of the mice. Significant infiltration was observed in liver, spleen and bone morrow in addition to parotid salivary glands, the eyes and harderrun gland related to the site of injection of the cells. Histological analyses of tumours grown in NSG mice showed the presence of large multinucleate CD30 positive cells as observed for human Hodgkin lymphoma. Of note, granuloma like Hodgkin lymphoma human lymph nodes were not observed because of the nature (immunodeficiency) of the mice used. In addition, because of the high number of cells injected, the survival of the mice did not exceed 6 weeks.

Unfortunately, no FDG up-take was observed in the liver tumour in mice. Some assays were performed using [18F] FDG PET/CT to image and quantify in vivo infiltration of tumour cells.

Despite this limitation in the PET/CT and given the high level of tumour infiltration in NSG mice, L428-c was considered to be the most suitable candidate for the establishment of this animal model for Hodgkin lymphoma and reproducibility and viability studies were performed.

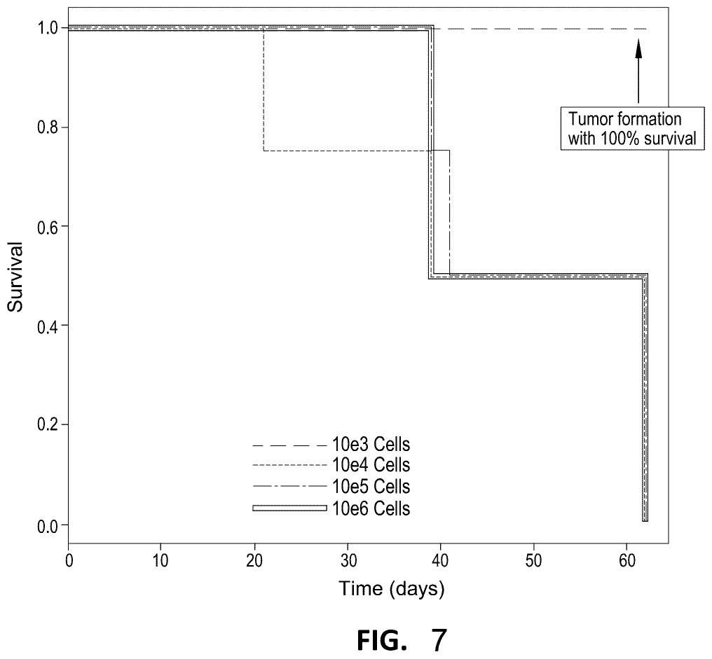

FIG. 7 shows the number of days of survival for this model after injection of from 10³ up to 10⁶ Hodgkin lymphoma cells, thus demonstrating the reproducibility of the model as well as the effect of reducing the number of injected cells, resulting in low toxicity and better compatibility with the viability of the mice.

### Example 9

### Antitumour Effects of EDO-S101 on NOD/SCID Gamma−/−Mice With Hodgkin Lymphoma Cell Line Xenografts

### Experimental Procedure

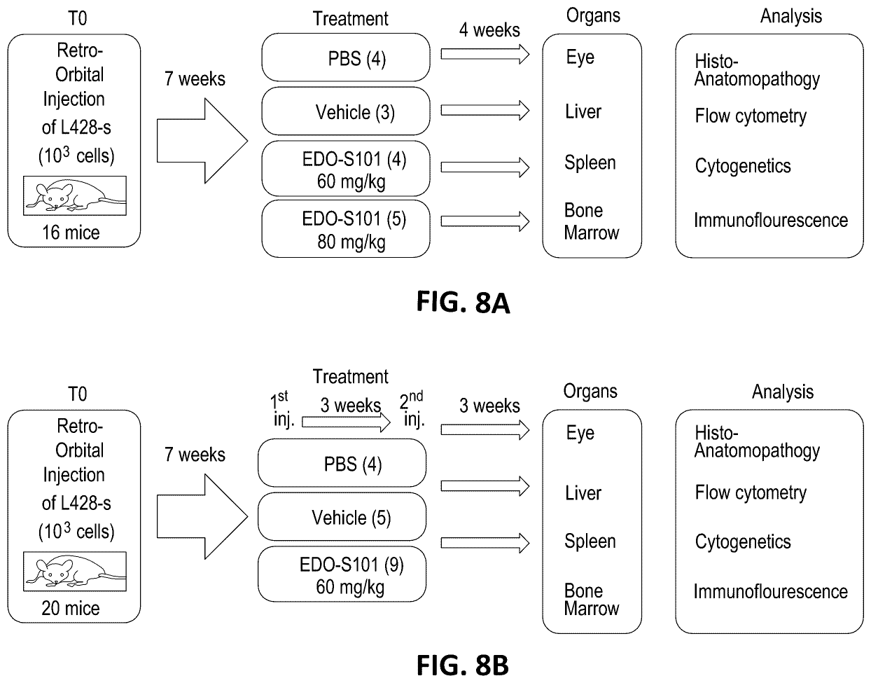

Two independent experiments were performed to investigate the dose-effect of EDO-S101 in NSG mice eight weeks after xenografting with L428 cells as in Example 8. Two different doses were tested, 60 mg/kg and 80 mg/kg, according to the protocol shown in FIG. 8A. The aim of the protocol shown in FIG. 8B was to evaluate the effect of sequential treatment with a moderate dose of 60 mg/kg given at an interval of three weeks.

The mice were sacrificed by cervical dislocation. The sampled organs consisted of the eyes (site of injection), liver, spleen and bone marrow. Following dissection of different organs, including tumours, the first portion was used for flow cytometry (FACS) and cytogenetic analyses while the second was fixed in 4% paraformaldehyde (PFA) for immunohistochemistry (IHC). To obtain a single cell suspension, the tissues were first cut into pieces. Thereafter, the cells were physically disaggregated using an 80 μm nylon cell strainer and filtered a second time through a 70 μm nylon cell strainer (BD Biosciences, Erembodegem, Belgium).

The organs were conducted to histophathological analysis, flow cytometry analyses and immunofluorescence and cytogenetic analyses according to the following procedures.

Immunohistochemistry analysis

Organs were fixed in 4% PFA, trimmed and post-fixed in 70° ethanol. They were then briefly processed using a vacuum inclusion processor and paraffin blocks were prepared. Five micrometer-thick sections were cut from these blocks and the resulting sections were stained with hemotoxylin-eosine (H&E) for histopathological analysis. In addition, IHC was carried out on of the totality of liver and spleen specimens for confirmation of the spread of engrafted cells. The search for single cells or small groups of grafted Hodgkin lymphoma cells was performed using an anti-CD30 antibody (DAKO, France; 1:40, EDTA pH9 pre-treatment) and the Ventana Discovery XT IHC system. Apoptosis and tumour necrosis were analysed.

Microscopic findings were assigned a severity score based upon the following scale: “0”=within physiological limits, “1”=minimal, “2”=mild, “3”=moderate, “4”=marked, “5”=severe. This severity score was attributed according to the size and number of observed lesions.

### Flow Cytometry

Cells were gated to exclude apoptotic or necrotic cells and sorted into CD30631 /CD15− and CD30+ and CD15+/CD30+ fractions by gating on the lowest and highest 5% PE-expressing cells, respectively. Following sorting, the CD30−/CD15− cell fractions were analyzed using a FACScan flow cytometer (Becton Dickinson) and found to be more than 98% pure. For phenotypic analyses of cell lines or sorted cells, cells were prepared as described, and then stained with mouse anti-human CD30-phycoerthrin (PE), CD15-fluorescein isothyocyanate (FITC), CD14-APC-cyanine 7 (Cy7) and CD45-APC (all antibodies from BD PharMingen, San Diego, CA). Staining for aldehyde dehydrogenase (ALDH) activity was performed using the Aldefluor reagent (StemCell Technologies, Vancouver, BC) according to the manufacturer's instructions. Cells were subsequently analysed using a FACS LSRII (Becton Dickinson, Franklin Lakes, NJ). The injected Hodgkin lymphoma L428-s cell line served as a control for all experiments.

Immunofluorescence for measuring CD30

Cells were cytospun onto poly-L-lysine-coated glass slides at 700 rpm for 4 min, fixed with 10% formalin for 10 min, and treated with 0.25% Triton X-100 solution for 10 min. After blocking with 5% bovine serum albumin (Sigma), the cells were incubated with an anti-CD30 antibody (DAKO A/S, Glostrup, Denmark). Then, cells were treated with Cyanine 3 anti-mouse lgG (Invitrogen, Carlsbad, CA). As a negative control, staining was carried out in the absence of primary antibody. The L428-s cell line served as positive control.

### Cytogenetic Analysis

Separated cells were cultured in the presence of RPMI 1640 supplemented with 10% Fetal Calf Serum (FCS) and antibiotics, colcemid (0.1 μg/ml) added 2h before harvesting, and the slides with metaphase chromosomes prepared following the standard methanol/acetic acid (3/1, v/v) procedure.

Four new cell lines were generated after one month of in vitro culture following 24 hour of exposure to EDO-S101. Fluorescence in situ hybridysation (FISH) was performed using a combination of standard procedures from the recommended protocols for chromosomal analysis, following telomere and centromere staining, using peptide nucleic acid (PNA) probes in order to detect unstable chromosomal aberrations and to delimit the chromosome territory. M-FISH was performed using multi-FISH probes (Metasystems GmbH, Altlussheim, Germany), according to the manufacturer's recommendations on the same slide. Images of hybridized metaphases were captured using a charge coupled device camera (Zeiss, Thornwood, NY) coupled to a Zeiss Axioplan microscope and processed using ISIS software (Metasystems).

### Results

(i) Phenotype of Mice Treated with EDO-S101

### (a) Effect of EDO-S101 Administration on the Weight of the Mice

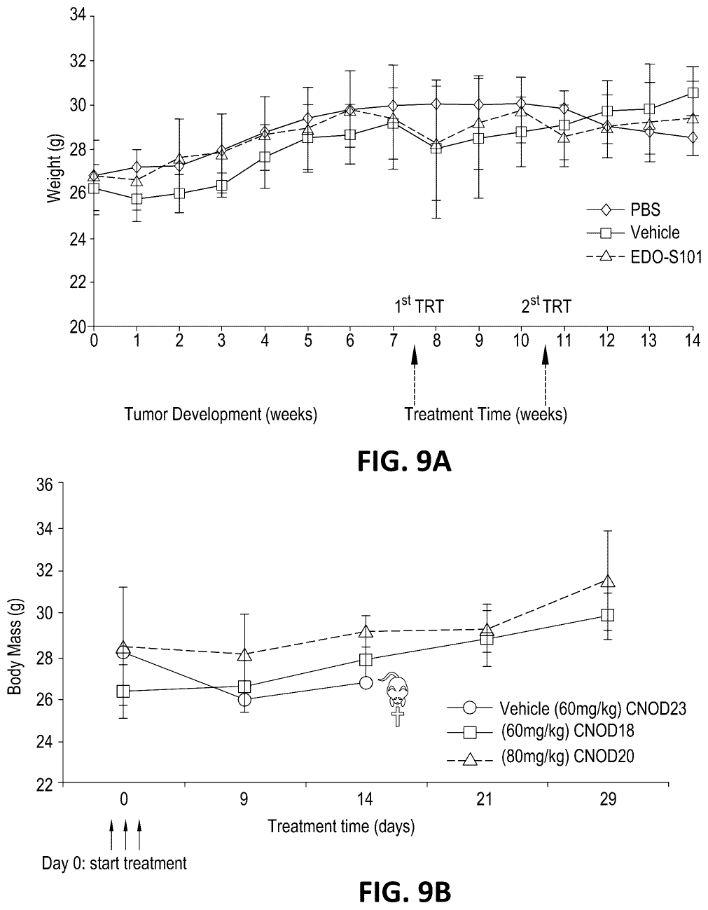

The evaluation of the mice treated sequentially with two doses of 60 mg/kg EDO-S101 three weeks apart demonstrated weight loss following each administration which was regained after several weeks (see FIG. 9A). This weight loss was also observed in mice treated with the vehicle. In contrast, the weight of the control mice receiving only freshly prepared phosphate buffered saline (PBS) buffered to 7.3-7.4 increased up to 11 weeks and then started to decrease.

The toxicity of the dose of 80 mg/kg compared to 60 mg/kg was investigated, as compared to the control groups vehicle and PBS. As shown in FIG. 9B, no significant difference was observed between the weight losses following treatment with 60 mg/kg or 80 mg/kg whereas mice treated with vehicle did not survive beyond 14 days.

### (b) Regression of the Orbital Tumour Mass Following EDO-S101 Treatment

Some mice developed an orbital tumour mass at the site of the injection. After EDO-S101injection, signification regression of this tumour was observed. In one mouse only that was treated with two doses of 60 mg/kg of EDO-S101, a regression of the orbital tumour was initially observed followed by re-establishment of the orbital mass 3 weeks later. However, this was the only instance observed of re-establishment of the orbital mass after treatment with EDO-S101 at either concentration.

(ii) Effect of dose

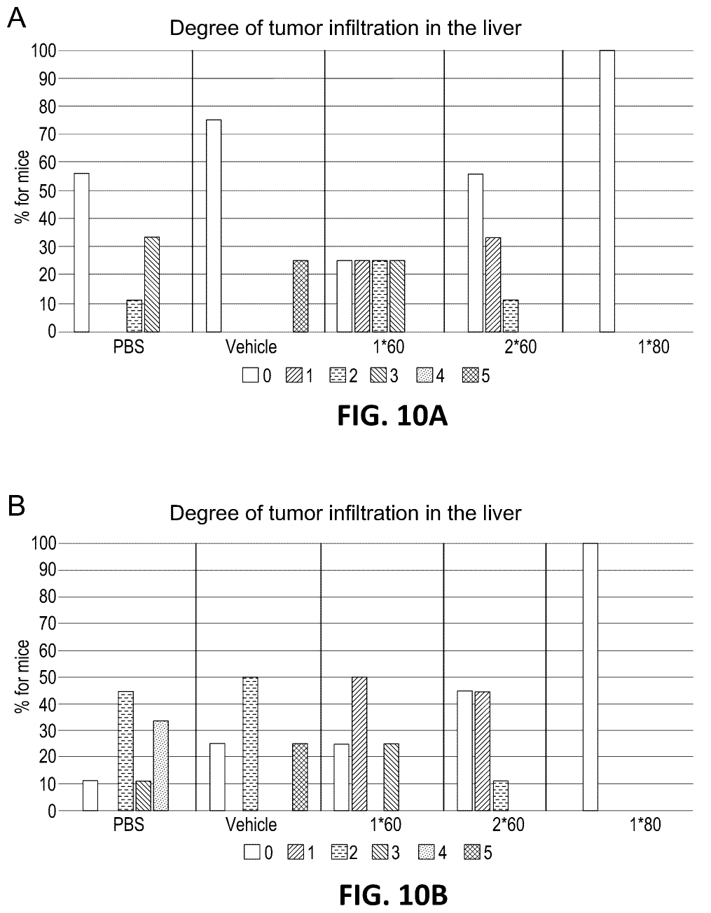

Tables 4, 5, 6 and 7 summarise the results obtained for the different treatments and doses as well as the analysed organs and the lesions detections observed following immunohistochemistry and immunofluorescence using CD30. No tumour masses were observed in the liver, nor was infiltration observed into the other organs following treatment with a dose of 80 mg/kg EDO-S101. FIG. 10A shows the absence of tumour cells in the liver following treatment with a single dose of 80 mg/kg by immunohistochemistry after HE staining. FIG. 10B shows the absence of tumour cells in the liver following treatment with a single dose of 80 mg/kg by immunofluorescence using CD30 on cytospin slides. No tumour cells were observed in any organs (liver, spleen, bone marrow).

Even after treatment with a single dose of 60 mg/kg, evidence of significant tumour regression was observed. Only one mouse had moderate tumour lesions, while the other mice had either no tumours or minimal lesions.

Individual microscopic findings following immunohistochemistry: “0”=within physiological limits, “1”=minimal, “2”=mild, “3”=moderate, “4”=marked, “5”=severe.

TABLE 4

| | | | Liver, Tumor cell | Liver, Tumor | Tumor tissue, |
|---|---|---|---|---|---|
| Histology | Animal | | infiltration, | cell necrosis/ | without connection |
| number | number | Group | focal/multifocal | Apoptosis | to other organs |
| 14-056 | cnod 10 | PBS | 0 | | |
| 14-057 | cnod 11 | PBS | 4 | 1 | |
| 14-058 | cnod 13 | PBS | 3 | 1 | |
| 14-059 | cnod 14 | PBS | 0 | | |
| 14-066 | cnod 23 | PBS | 4 | 3 | yes |
| 14-083 | cnod 31 | PBS | 4 | 2 | yes |
| 14-085 | cnod 34 | PBS | 0 | | |
| 14-086 | cnod 36 | PBS | 0 | | yes |
| 14-106 | cnod 46 | PBS | 0 | | |
| 14-084 | cnod 32 | Veh | 5 | 5 | |
| 14-087 | cnod 37 | Veh | 0 | | |
| 14-102 | cnod 38 | Veh | 0 | | |
| 14-103 | cnod 39 | Veh | 0 | | |
| 14-060 | cnod 16 | 1*60 mg/kg | 2 | 0 | |
| 14-061 | cnod 18 | 1″60 mg/kg | 1 | 0 | |
| 14-062 | cnod 19 | 1″60 mg/kg | 0 | | |
| 14-067 | cnod 17 | 1*60 mg/kg | 3 | 0 | |
| 14-063 | cnod 20 | 1*80 mg/kg | 0 | | |
| 14-064 | cnod 21 | 1*80 mg/kg | 0 | | |
| 14-065 | cnod 22 | 1″80 mg/kg | 0 | | |
| 14-088 | cnod 41 | 2*60 mg/kg | 0 | | |
| 14-089 | cnod 42 | 2*60 mg/kg | 0 | | |
| 14-090 | cnod 45 | 2*60 mg/kg | 1 | 0 | yes |
| 14-091 | cnod 49 | 2*60 mg/kg | 0 | | |
| 14-104 | cnod 43 | 2*60 mg/kg | 0 | | |
| 14-105 | cnod 44 | 2*60 mg/kg | 1 | 0 | |
| 14-107 | cnod 47 | 2*60 mg/kg | 0 | | |
| 14-108 | cnod 48 | 2*60 mg/kg | 1 | 0 | yes |
| 14-109 | cnod 50 | 2*60 mg/kg | 2 | 0 | |

| | Eye and/or | | | | Kidney, cortex, |
|---|---|---|---|---|---|
| | periocular mucosa, | Tumor/Eye, | | Spleen, | tubular degeneration, |
| | periocular tumor | Tumor cell | | red pulp | vacuolar, acute, |
| Histology | cell infiltration, | necrosis/ | Lung, alveolar | activation, | bilateral focal/ |
| number | focal/multifocal/diffuse | appotosis | histiocytosis | diffuse | multifocal/diffuse |
| 14-056 | | | | | |
| 14-057 | | | | | |
| 14-058 | | | 2 | 2 | |
| 14-059 | | | | | |
| 14-066 | | 4 | | 1 | 3 |
| 14-083 | | 3 | | | |
| 14-085 | | | | | |
| 14-086 | | 3 | | 1 | |
| 14-106 | | | | | |
| 14-084 | | | | 2 | |
| 14-087 | | | | | |
| 14-102 | 1 | 0 | | 2 | |
| 14-103 | | | | | |
| 14-060 | 5 | 0 | | | |
| 14-061 | 4 | 0 | | | |
| 14-062 | 3 | 1 | 1 | | |
| 14-067 | 4 | 1 | | | |
| 14-063 | | | | | |
| 14-064 | 5 | 1 | | | |
| 14-065 | | | 1 | | |
| 14-088 | | | | | |
| 14-089 | | | | | |
| 14-090 | | 4 | | | |
| 14-091 | 4 | 2 | | | |
| 14-104 | | | | | |
| 14-105 | | | | 3 | |
| 14-107 | | | | | |
| 14-108 | | 2 | | | |
| 14-109 | | | | | |

Incidence of tumour cell infiltration using HE staining and immunohistochemistry

TABLE 5

| | | | | | 1*60 | | 2*60 | | 80 | |
|---|---|---|---|---|---|---|---|---|---|---|
| | PBS | | Veh | | mg/kg | | mg/kg | | mg/kg | |
| | n | % | n | % | n | % | n | % | n | % |
| Total number | 9 | 100.0 | 4 | 100.0 | 4 | 100.0 | 9 | 100.0 | 5 | 100.0 |
| of mice | | | | | | | | | | |

Tumour presence

| | | | | | | | | | | |
|---|---|---|---|---|---|---|---|---|---|---|
| Overall | 5 | 55.6 | 2 | 50.0 | 4 | 100.0 | 5 | 55.6 | 1 | 33.3 |
| Animals with | 2 | 22.2 | 0 | 0.0 | 3 | 75.0 | 0 | 0.0 | 0 | 0.0 |
| tumour infiltration | | | | | | | | | | |
| in liver and in the | | | | | | | | | | |
| injection area | | | | | | | | | | |

Liver: Tumour infiltration

| | | | | | | | | | | |
|---|---|---|---|---|---|---|---|---|---|---|
| Overall | 4 | 44.4 | 1 | 25.0 | 3 | 75.0 | 4 | 44.4 | 0 | 0.0 |
| No | 5 | 55.6 | 3 | 75.0 | 1 | 25.0 | 5 | 55.6 | 3 | 100.0 |
| Minimal | 0 | 0.0 | 0 | 0.0 | 1 | 25.0 | 3 | 33.3 | 0 | 0.0 |
| Mild | 0 | 0.0 | 0 | 0.0 | 1 | 25.0 | 1 | 11.1 | 0 | 0.0 |
| Moderate | 1 | 11.1 | 0 | 0.0 | 1 | 25.0 | 0 | 0.0 | 0 | 0.0 |
| Marked | 3 | 33.3 | 0 | 0.0 | 0 | 0.0 | 0 | 0.0 | 0 | 0.0 |
| Severe | 0 | 0.0 | 1 | 25.0 | 0 | 0.0 | 0 | 0.0 | 0 | 0.0 |

Liver: Tumour cell necrosis/apoptosis

| | | | | | | | | | | |
|---|---|---|---|---|---|---|---|---|---|---|
| Overall | 4 | 100.0 | 1 | 100.0 | 0 | 0.0 | 0 | 0.0 | NA | NA |
| No | 0 | 0.0 | 0 | 0.0 | 3 | 100.0 | 4 | 100.0 | NA | NA |
| Minimal | 2 | 50.0 | 0 | 0.0 | 0 | 0.0 | 0 | 0.0 | NA | NA |
| Mild | 1 | 25.0 | 0 | 0.0 | 0 | 0.0 | 0 | 0.0 | NA | NA |
| Moderate | 1 | 25.0 | 0 | 0.0 | 0 | 0.0 | 0 | 0.0 | NA | NA |
| Marked | 0 | 0.0 | 0 | 0.0 | 0 | 0.0 | 0 | 0.0 | NA | NA |
| Severe | 0 | 0.0 | 1 | 100.0 | 0 | 0.0 | 0 | 0.0 | NA | NA |

Injection site: Tumour infiltration

| | | | | | | | | | | |
|---|---|---|---|---|---|---|---|---|---|---|
| Overall | 3 | 33.3 | 1 | 25.0 | 4 | 100.0 | 1 | 11.1 | 1 | 33.3 |
| No | 0 | 0.0 | 0 | 0.0 | 0 | 0.0 | 0 | 0.0 | 0 | 0.0 |
| Minimal | 0 | 0.0 | 1 | 25.0 | 0 | 0.0 | 0 | 0.0 | 0 | 0.0 |
| Mild | 0 | 0.0 | 0 | 0.0 | 0 | 0.0 | 0 | 0.0 | 0 | 0.0 |
| Moderate | 0 | 0.0 | 0 | 0.0 | 1 | 25.0 | 0 | 0.0 | 0 | 0.0 |
| Marked | 0 | 0.0 | 0 | 0.0 | 2 | 50.0 | 1 | 11.1 | 0 | 0.0 |
| Severe | 3 | 33.3 | 0 | 0.0 | 1 | 25.0 | 0 | 0.0 | 1 | 33.3 |

Injection site: Tumour cell necrosis/apoptosis

| | | | | | | | | | | |
|---|---|---|---|---|---|---|---|---|---|---|
| Overall | 3 | 100.0 | 0 | 0.0 | 4 | 100.0 | 3 | 100.0 | 1 | 100.0 |
| No | 0 | 0.0 | 1 | 100.0 | 2 | 50.0 | 0 | 0.0 | 0 | 0.0 |
| Minimal | 0 | 0.0 | 0 | 0.0 | 2 | 50.0 | 0 | 0.0 | 0 | 0.0 |
| Mild | 0 | 0.0 | 0 | 0.0 | 0 | 0.0 | 2 | 66.7 | 0 | 0.0 |
| Moderate | 2 | 66.7 | 0 | 0.0 | 0 | 0.0 | 0 | 0.0 | 0 | 0.0 |
| Marked | 1 | 33.3 | 0 | 0.0 | 0 | 0.0 | 1 | 33.3 | 0 | 0.0 |
| Severe | 0 | 0.0 | 0 | 0.0 | 0 | 0.0 | 0 | 0.0 | 0 | 0.0 |

Individual microscopic findings following immunofluorescence CD30 staining of cytopsin slides: “0”=without any staining; “1”=minimal, <1% staining; “2”=mild, 1% staining; “3”=moderate, <10% staining; “4”=marked, 10-40% staining; “5”=severe, >40% of cells staining.

TABLE 6

| | | | Liver, | Eye and/or periocular | | Spleen, |
|---|---|---|---|---|---|---|
| | | | Tumour | mucosa, periocular | | red pulp |
| Histology | Animal | | cell | tumour cell infiltration, | Bone | activation, |
| number | number | Group | infiltration | focal/multifocal/diffuse | marrow | diffuse |
| 14-030 | cnod 10 | PBS | 2 | | 1 | 1 |
| 14-032 | cnod 11 | PBS | 4 | | 2 | 1 |
| 14-033 | cnod 13 | PBS | 3 | 3 | 2 | 2 |
| 14-024 | cnod 14 | PBS | 2 | | 1 | 1 |
| 14-023 | cnod 23 | PBS | 4 | 4 | 2 | 1 |
| 14-001 | cnod 31 | PBS | 4 | 3 | 1 | 0 |
| 14003 | cnod 34 | PBS | 2 | | 0 | 0 |
| 14-007 | cnod 36 | PBS | 2 | 3 | 1 | 1 |
| 14-013 | cnod 46 | PBS | 0 | | | |
| 14-002 | cnod 32 | Veh | 5 | | 2 | 2 |
| 14009 | cnod 37 | Veh | 0 | | | |
| 14-016 | cnod 38 | Veh | 2 | 3 | | 0 |
| 14-010 | cnod 39 | Veh | 2 | | 1 | 1 |
| 14-016 | cnod 16 | 1*60 mg/kg | 1 | 4 | | 0 |
| 14-021 | cnod 18 | 1*60 mg/kg | 1 | 4 | | |
| 14-019 | cnod 19 | 1*60 mg/kg | 0 | 3 | 1 | |
| 14-018 | cnod 17 | 1*60 mg/kg | 3 | 4 | | |
| 14-020 | cnod 20 | 1*80 mg/kg | 0 | | 0 | 0 |
| 14-021 | cnod 21 | 1*80 mg/kg | 0 | 2 | | |
| 14-022 | cnod 22 | 1*80 mg/kg | 0 | | 1 | |
| 14-006 | cnod 41 | 2*60 mg/kg | 0 | | 0 | |
| 14-005 | cnod 42 | 2*60 mg/kg | 0 | | | |
| 14-004 | cnod 45 | 2*60 mg/kg | 1 | | 1 | 0 |
| 14-008 | cnod 49 | 2*60 mg/kg | 1 | 2 | 1 | 1 |
| 14-011 | cnod 43 | 2*60 mg/kg | 0 | | 0 | |
| 14-012 | cnod 44 | 2*60 mg/kg | 1 | | 1 | 1 |
| 14-014 | cnod 47 | 2*60 mg/kg | 0 | | 1 | 1 |
| 14-015 | cnod 48 | 2*60 mg/kg | 1 | | | |
| 14-017 | cnod 50 | 2*60 mg/kg | 2 | | 1 | 1 |

Incidence of tumour cell infiltration following CD30 immunofluorescence staining of cytopsin slides.

TABLE 7

| | | | | | 1*60 | | 2*60 | | 80 | |
|---|---|---|---|---|---|---|---|---|---|---|
| | PBS | | Veh | | mg/kg | | mg/kg | | mg/kg | |
| | n | % | n | % | n | % | n | % | n | % |
| Total number | 9 | 100.0 | 4 | 100.0 | 4 | 100.0 | 9 | 100.0 | 3 | 100.0 |
| of mice | | | | | | | | | | |

Tumour presence

| | | | | | | | | | | |
|---|---|---|---|---|---|---|---|---|---|---|
| Overall | 8 | 88.9 | 3 | 75.0 | 4 | 100.0 | 6 | 66.7 | 2 | 66.7 |

Liver: Tumour infiltration

| | | | | | | | | | | |
|---|---|---|---|---|---|---|---|---|---|---|
| Overall | 8 | 88.9 | 3 | 75.0 | 3 | 75.0 | 5 | 55.6 | 0 | 0.0 |
| No | 1 | 11.1 | 1 | 25.0 | 1 | 25.0 | 4 | 44.4 | 3 | 100.0 |
| Minimal | 0 | 0.0 | 0 | 0.0 | 2 | 50.0 | 4 | 44.4 | 0 | 0.0 |
| Mild | 4 | 44.4 | 2 | 50.0 | 0 | 0.0 | 1 | 11.1 | 0 | 0.0 |
| Moderate | 1 | 11.1 | 0 | 0.0 | 1 | 25.0 | 0 | 0.0 | 0 | 0.0 |
| Marked | 3 | 33.3 | 0 | 0.0 | 0 | 0.0 | 0 | 0.0 | 0 | 0.0 |
| Severe | 0 | 0.0 | 1 | 25.0 | 0 | 0.0 | 0 | 0.0 | 0 | 0.0 |

Spleen: Tumour infiltration

| | | | | | | | | | | |
|---|---|---|---|---|---|---|---|---|---|---|
| Overall | 6 | 66.7 | 2 | 50.0 | 0 | 0.0 | 4 | 44.4 | 0 | 0.0 |
| No | 2 | 22.2 | 1 | 25.0 | 1 | 25.0 | 1 | 11.1 | 1 | 33.3 |
| Minimal | 5 | 55.6 | 1 | 25.0 | 0 | 0.0 | 4 | 44.4 | 0 | 0.0 |
| Mild | 1 | 11.1 | 1 | 25.0 | 0 | 0.0 | 0 | 0.0 | 0 | 0.0 |
| Moderate | 0 | 0.0 | 0 | 0.0 | 0 | 0.0 | 0 | 0.0 | 0 | 0.0 |
| Marked | 0 | 0.0 | 0 | 0.0 | 0 | 0.0 | 0 | 0.0 | 0 | 0.0 |
| Severe | 0 | 0.0 | 0 | 0.0 | 0 | 0.0 | 0 | 0.0 | 0 | 0.0 |

Bone Marrow: Tumour infiltration

| | | | | | | | | | | |
|---|---|---|---|---|---|---|---|---|---|---|
| Overall | 7 | 77.8 | 2 | 50.0 | 1 | 25.0 | 5 | 55.6 | 1 | 33.3 |
| No | 1 | 11.1 | 0 | 0.0 | 0 | 0.0 | 2 | 22.2 | 1 | 33.3 |
| Minimal | 4 | 44.4 | 1 | 25.0 | 1 | 25.0 | 5 | 55.6 | 1 | 33.3 |
| Mild | 3 | 33.3 | 1 | 25.0 | 0 | 0.0 | 0 | 0.0 | 0 | 0.0 |
| Moderate | 0 | 0.0 | 0 | 0.0 | 0 | 0.0 | 0 | 0.0 | 0 | 0.0 |
| Marked | 0 | 0.0 | 0 | 0.0 | 0 | 0.0 | 0 | 0.0 | 0 | 0.0 |
| Severe | 0 | 0.0 | 0 | 0.0 | 0 | 0.0 | 0 | 0.0 | 0 | 0.0 |

Injection site: Tumour infiltration

| | | | | | | | | | | |
|---|---|---|---|---|---|---|---|---|---|---|
| Overall | 4 | 44.4 | 1 | 25.0 | 4 | 100.0 | 1 | 11.1 | 1 | 33.3 |
| No | 0 | 0.0 | 0 | 0.0 | 0 | 0.0 | 0 | 0.0 | 0 | 0.0 |
| Minimal | 0 | 0.0 | 0 | 0.0 | 0 | 0.0 | 0 | 0.0 | 0 | 0.0 |
| Mild | 0 | 0.0 | 0 | 0.0 | 0 | 0.0 | 1 | 11.1 | 1 | 33.3 |
| Moderate | 3 | 33.3 | 1 | 25.0 | 1 | 25.0 | 0 | 0.0 | 0 | 0.0 |
| Marked | 1 | 11.1 | 0 | 0.0 | 3 | 75.0 | 0 | 0.0 | 0 | 0.0 |
| Severe | 0 | 0.0 | 0 | 0.0 | 0 | 0.0 | 0 | 0.0 | 0 | 0.0 |

These results clearly show the effect of single doses of EDO-S101 at both 60 mg/kg and 80 mg/kg in comparison with the control (PBS or vehicle). There was a remarkable effect on the tumour cell infiltration. No tumour masses were observed in the liver, nor was infiltration observed into the other organs following treatment at the higher dose of 80 mg/kg EDO-S101. At the lower dose of 60 mg/kg only one mouse had moderate tumour lesions, while the other mice had either no tumours or minimal lesions.

Evaluation of tumour cell degeneration (necrosis/apoptosis) based on morphology of the cells was also performed as part of the histopathological evaluation. No cell necerosis was observed following treatment with either 80 mg/kg or 60 mg/kg of EDO-S101. As can be seen from Tables 4 and 5, the PBS control group presented larger tumours compared to the 60 mg/kg EDO-S101 group and they showed a higher degree of necrosis/apoptosis.

A study of the effect of sequential treatment with two doses of 60 mg/kg EDO-S101 three weeks apart was also performed. Significant tumour regression was observed following two administrations of 60 mg/kg EDO-S101 compared with the control group and with the single dose of 60 mg/kg EDO-S101 (see FIGS. 10A and 10B). Only one mouse exhibited a moderate level of infiltration in the liver. No tumours were observed in any organs (liver, spleen, bone marrow), as can be seen from Tables 4 to 7.

(iii) Cytogenic Investigations

The ocular tumour masses were harvested, as well as cells which infiltrated the liver. Five new cell lines were established from ocular tumours and the livers of both control mice (PBS or vehicle). Cytogenic characterisation using the M-FISH technique was performed and the karyotype was established in L428, L428-s and in new cell lines established from liver tumours of the mice. The presence of small (<46 chromosomes) metaphases was found only in L428-s and cell lines established from liver tumours of the mice. Typical chromosomal abnormalities were found in the cell lines.

For treated mice, it was not possible to obtain either metaphases from direct culture or establish cell lines following the culture of mouse cells treated with different doses of EDO-S101, including a single dose of 60 mg/kg. The few cells present on cytogenic slides showed centromere staining that was abnormal, which could be an indicator of their poor viability.

### (iv) Presence of Macrophages

Novel biomarkers CD68 and CD14 which are expressed by tumour associated macrophages and myeloid derived suppressor cells respectively in the microenvironment have recently been reported to affect the prognosis of Hodgkin lymphoma (Haematologica, 2011 February; 96(2): 186-189). The presence of the CD14-positive cells following administration of a single dose of EDO-S101 (60 mg/kg or 80 mg/kg) and after two administrations of EDO-S101 at 60 mg/kg was evaluated using flow cytometry with anti CD14 antibodies.

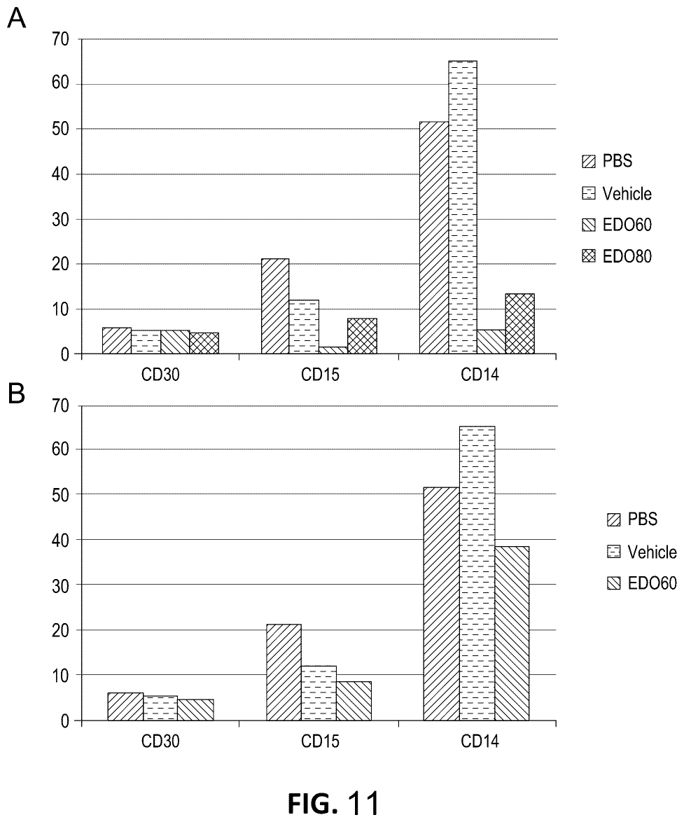

FIG. 11 shows the significantly higher frequency of CD14 cells in untreated mice compared to treated mice, especially at the higher dose of 80 mg/kg EDO-S101. Graph A of FIG. 11 shows the frequency of CD14 cells after treatment with a single dose of EDO-S101 at 60 mg/kg while graph B shows the frequency of CD14 cells after two administrations of EDO-S101 of EDO-S101 at 60 mg/kg. These CD14 cells cells were found at a higher frequency following treatment with 2×60 mg/kg than treatment with a single dose of 80 mg/kg.

The in vitro results demonstrate clearly the sensitivity of all Hodgkin lymphoma cell lines used to treatment with the compound of formula I according to the present invention. This sensitivity implicates different mechanisms including chromosomal aberrations, micronucleus and protein expression.

These in vitro experiments demonstrate that monotherapy with the compound of formula I or a pharmaceutically acceptable salt thereof of the present invention displays a striking time and dose-dependent cytotoxic activity towards all Hodgkin lymphoma cell lines. The IC50s of the compound of formula I or a pharmaceutically acceptable salt thereof of the present invention are about ten-fold lower than those of bendamustine towards tumour cells of Hodgkin lymphoma. In bendamustine-naïve Hodgkin lymphoma cells (L1236) the compound of formula I or a pharmaceutically acceptable salt thereof of the present invention has been demonstrated to activate cell cycle check point proteins and induce cellular and gene expression changes compatible with triggering of apoptosis. In bendamustine-resistant Hodgkin lymphoma cells (L1236 R100) the compound of formula I or a pharmaceutically acceptable salt thereof of the present invention has been demonstrated to downregulate activated cell cycle checkpoints and mitotic catastrophe genes and upregulate proapoptotic genes. It also corrects the constitutive ATM/ATR unbalance by upregulating ATR and lowering ATM transcripts in bendamustine-resistant Hodgkin lymphoma cells L1236 R100.

These results are backed up by the in vivo results in the NOD/SCID mice xenografted with the Hodgkin lymphoma cell line 428-s. Using either a single injection of the compound of formula I (60 mg/kg or 80 mg/kg) or two injections three weeks apart (each at 60 mg/kg) a significant decrease in the severity of tumoural cell infiltration after treatment was observed, especially at the single dose of 80 mg/kg (although all dosage regimes were effective). All mice were observed to determine the inter-mouse variation with respect to the organ and the level of infiltration, but in all treated mice a high level of infiltration was not observed demonstrating the efficacy of the compound of formula I or a pharmaceutically acceptable salt thereof.

The absence of necrotic cells in treated mice and the absence of cell degeneration compared to the situation observed in untreated mice could be correlated with the anti-tumoural effect of the compound of formula I or a pharmaceutically acceptable salt thereof. The necrotic cells observed in untreated mice could be related to spontaneous apoptosis. No significant difference was observed for the weight of the mice or the different organs. Additionally, the weight of the liver in the different mice shows a high degree of heterogeneity in the control mice (PBS or vehicle) but the liver remained homogeneous in the mice treated with the compound of formula I or a pharmaceutically acceptable salt thereof.

In conclusion, the compound of formula I or a pharmaceutically acceptable salt thereof according to the present invention is a very active molecule in these in vitro and in vivo pre-clinical models of Hodgkin lymphoma. This includes tumour cells that have previously been very difficult to treat including bendamustine-resistant Hodgkin lymphomas such as the L1236 R100 cell line. The compound of formula I or a pharmaceutically acceptable salt thereof of the present invention has unique and particularly effective combined function in a single molecule of a bifunctional alkylator and a pan-HDAC inhibitor. Following a strong DNA-damage response, triggering of apoptosis and/or mitotic catastrophe may take place in Hodgkin lymphoma cells according to their sensitivity to bendamustine. The compound of formula I or a pharmaceutically acceptable salt thereof represents an agent of great potential advantage in the treatment of Hodgkin lymphoma based on the presented data.

### Example 10

### Combination Therapy with Brentuximab Vedotin

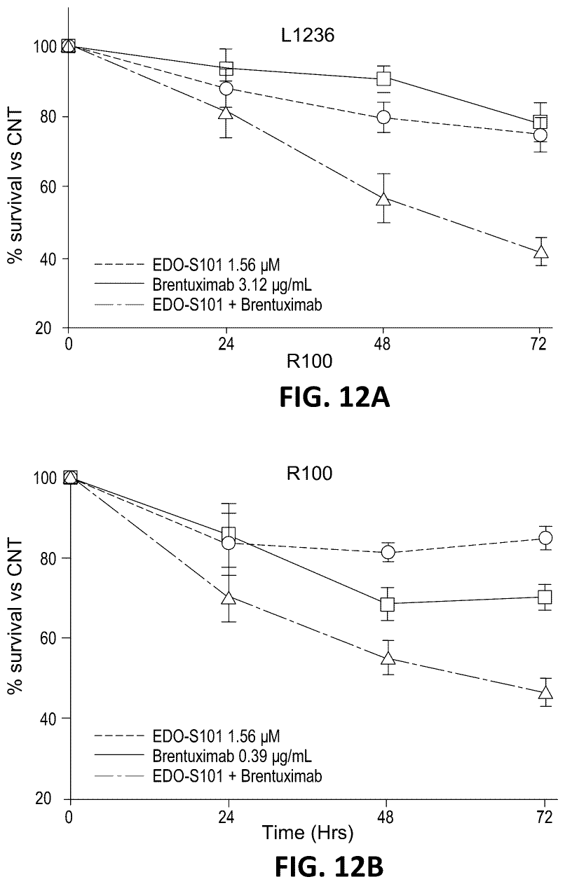

1.56 μM of EDO-S101 was administered to two Hodgkin lymphoma cell lines, the bendamustine naïve cell line L1236 and the bendamustine resistant cell line R100. In a second experiment, Brentuximab Vedotin was administered at a concentration of 3.12 μg/ml to the L1236 cell line and at a concentration of 0.39 μg/ml to the R100 cell line. Finally, in a third experiment EDO-S101 and Brentuximab Vedotin were administered in combination to each of the two cell lines at the same concentrations that they were administered in the monotherapy experiments. The results showing % survival of the lymphoma cell lines against a control versus times are as shown in FIGS. 12A and 12B.

In the naïve L1236 cell line, EDO-S101 alone activated cell cycle checkpoint proteins and induced cellular and gene expression changes compatible with the triggering of apoptosis. In the bendamustine resistant R100 cell line, EDO-S101 alone was found to correct the constitutive ATM/ATR unbalance by upregulating ATR and downregulating ATM transcripts.

Most importantly, it was found that EDO-S101 at sub-IC50 levels is synergistic with Brentuximab Vedotin in inducing cell death of L1236 cells and furthermore that at sub-IC concentrations EDO-S101 allows low doses of Brentuximab Vedotin (typically 10-fold lower than IC50) to exert a considerable cyctotoxic effect on bendamustine resistant R100 cells.

1. A compound of formula I or a pharmacologically acceptable salt thereof for use in a method of treating Hodgkin lymphoma in a patient in need thereof comprising administering to said patient an effective amount of said compound of formula I or a pharmacologically acceptable salt thereof:

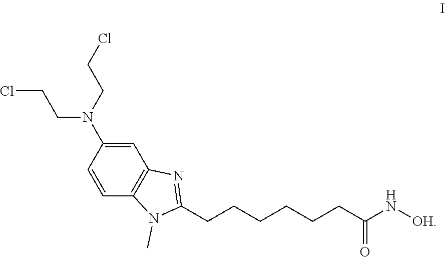 [SMILES: Cn1c(CCCCCCC(=O)NO)nc2cc(N(CCCl)CCCl)ccc21]

2. The compound of formula I or a pharmacologically acceptable salt thereof for use in a method of treating Hodgkin lymphoma according to claim 1, wherein the pharmacologically acceptable salt of the compound of formula I is the hydrochloride, hydrobromide, hydroiodide, sulfate, bisulfate, sulfamate, nitrate, phosphate, citrate, methanesulfonate, trifluoroacetate, glutamate, glucuronate, glutarate, malate, maleate, oxalate, succinate, fumarate, tartrate, tosylate, mandelate, salicylate, lactate, p-toluenesulfonate, naphthalenesulfonate or acetate.

3. The compound of formula I or a pharmacologically acceptable salt thereof for use in a method of treating Hodgkin lymphoma according to claim 1, wherein the pharmacologically acceptable salt of the compound of formula I is the acetate.

4. The compound of formula I or a pharmacologically acceptable salt thereof for use in a method of treating Hodgkin lymphoma according to any one of claims 1 to 3, wherein the compound of formula I or a pharmaceutically acceptable salt thereof is not administered in combination with a compound selected from the group consisting of proteasome inhibitors, glucocorticoids and class III receptor tyrosine kinase inhibitors.

5. The compound of formula I or a pharmacologically acceptable salt thereof for use in a method of treating Hodgkin lymphoma according to any one of claims 1 to 4, wherein the method of treating Hodgkin lymphoma is a monotheraputic treatment consisting of the administration of the compound of formula I or a pharmacologically acceptable salt thereof to the patient in need thereof.

6. The compound of formula I or a pharmacologically acceptable salt thereof for use in a method of treating Hodgkin lymphoma according to any one of claims 1 to 5, wherein the Hodgkin lymphoma is classical Hodgkin lymphoma or lymphocyte predominant Hodgkin lymphoma.

7. The compound of formula I or a pharmacologically acceptable salt thereof for use in a method of treating Hodgkin lymphoma according to any one of claims 1 to 5, wherein the Hodgkin lymphoma is nodular sclerosis classical Hodgkin lymphoma, mixed cellularity classical Hodgkin lymphoma, lymphocyte-depletion classical Hodgkin lymphoma or lymphocyte-rich classical Hodgkin lymphoma.

8. The compound of formula I or a pharmacologically acceptable salt thereof for use in a method of treating Hodgkin lymphoma according to any one of claims 1 to 5, wherein the Hodgkin lymphoma is nodular lymphocyte predominant Hodgkin lymphoma.

9. The method according to any one of claims 1 to 8, wherein the Hodgkin lymphoma is relapsed/refractory Hodgkin lymphoma.

10. The compound of formula I or a pharmacologically acceptable salt thereof for use in a method of treating Hodgkin lymphoma according to any one of claims 1 to 9, wherein the compound of formula I or a pharmacologically acceptable salt thereof is administered intravenously to the patient in need thereof at a dosage level of from 0.3 to 100 mg/m² body surface area of the patient.

11. The compound of formula I or a pharmacologically acceptable salt thereof for use in a method of treating Hodgkin lymphoma according to any one of claims 1 to 9, wherein the compound of formula I or a pharmacologically acceptable salt thereof is administered intravenously to the patient in need thereof at a dosage level of from 20 mg/m² to 150 mg/m² body surface area of the patient or 40 mg/m² to 100 mg/m² body surface area of the patient.

12. The compound of formula I or a pharmacologically acceptable salt thereof for use in a method of treating Hodgkin lymphoma according to any one of claims 1 to 9, wherein the compound of formula I or a pharmacologically acceptable salt thereof is administered intravenously to the patient in need thereof at a dosage level of 80 mg/m² body surface area of the patient.

13. The compound of formula I or a pharmacologically acceptable salt thereof for use in a method of treating Hodgkin lymphoma according to any one of claims 1 to 9, wherein the compound of formula I or a pharmacologically acceptable salt thereof is administered intravenously to the patient in need thereof at a dosage level of 60 mg/m² body surface area of the patient.

14. The compound of formula I or a pharmacologically acceptable salt thereof for use in a method of treating Hodgkin lymphoma according to any one of claims 1 to 13, wherein the compound of formula I or a pharmacologically acceptable salt thereof is administered intravenously to the patient in need thereof on days 1, 8 and 15 of a treatment cycle, for 4 to 6 weeks, followed by a break of 1 to 3 weeks.

15. The compound of formula I or a pharmacologically acceptable salt thereof for use in a method of treating Hodgkin lymphoma according to any one of claims 1 to 13, wherein the compound of formula I or a pharmacologically acceptable salt thereof is administered intravenously to the patient in need thereof on days 1 and 22 of the treatment cycle, for 3 to 12 cycles of treatment, preferably 5 to 8 cycles, and most preferably 6 cycles.

16. The compound of formula I or a pharmacologically acceptable salt thereof for use in a method of treating Hodgkin lymphoma according to any one of claims 1 to 13, wherein the compound of formula I or a pharmacologically acceptable salt thereof is administered intravenously to the patient in need thereof on days 1 and 8 of the treatment cycle, and this is repeated for 4 cycles.

17. The compound of formula I or a pharmacologically acceptable salt thereof for use in a method of treating Hodgkin lymphoma according to any one of claims 1 to 13, wherein the compound of formula I or a pharmacologically acceptable salt thereof is administered intravenously to the patient in need thereof on days 1 and 15 of the treatment cycle, and this is repeated for 4 cycles.

18. The compound of formula I or a pharmacologically acceptable salt thereof for use in a method of treating Hodgkin lymphoma according to any one of claims 1 to 17, wherein the compound of formula I or a pharmacologically acceptable salt thereof is administered intravenously to the patient in need thereof at a dosage level of from 40 mg/m² to 100 mg/m² body surface area of the patient on days 1, 8 and 15 of a treatment cycle, for 4 to 6 weeks, followed by a break of 1 to 3 weeks.

19. The compound of formula I or a pharmacologically acceptable salt thereof for use in a method of treating Hodgkin lymphoma according to any one of claims 1 to 17, wherein the compound of formula I or a pharmacologically acceptable salt thereof is administered intravenously to the patient in need thereof at a dosage level of from 40 mg/m² to 100 mg/m² body surface area of the patient on days 1 and 22 of a treatment cycle, for 3 to 12 consecutive cycles, more preferably 5 to 8 cycles and most preferably 6 cycles.

20. The compound of formula I or a pharmacologically acceptable salt thereof for use in a method of treating Hodgkin lymphoma according to any one of claims 1 to 17, wherein the compound of formula I or a pharmacologically acceptable salt thereof is administered intravenously to the patient in need thereof at a dosage level of 40 mg/m² to 100 mg/m² body surface area of the patient on days 1 and 15 of a treatment cycle, for 4 consecutive cycles.

21. The compound of formula I or a pharmacologically acceptable salt thereof for use in a method of treating Hodgkin lymphoma according to any one of claims 1 to 17, wherein the compound of formula I or a pharmacologically acceptable salt thereof is administered intravenously to the patient in need thereof at a dosage level of 40 mg/m² to 100 mg/m² body surface area of the patient on days 1 and 8 of a treatment cycle, for 4 consecutive cycles.

22. The compound of formula I or a pharmacologically acceptable salt thereof for use in a method of treating Hodgkin lymphoma according to any one of claims 1 to 21, wherein the patient in need thereof is given radiotherapy prior to or after treatment of the Hodgkin lymphoma with the compound of formula I or a pharmacologically acceptable salt thereof.

23. The compound of formula I or a pharmacologically acceptable salt thereof for use in a method of treating Hodgkin lymphoma according to claim 22, wherein said radiotherapy treatment is given to the patient in need thereof at a dose of 1 to 5 Gy over 5 consecutive days, and preferably 2 Gy over 5 consecutive days.

24. A combination comprising Brentuximab Vedotin and a compound of formula I or a pharmaceutically acceptable salt thereof:

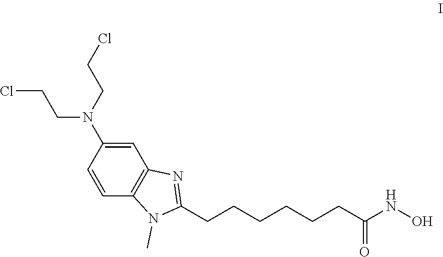 [SMILES: Cn1c(CCCCCCC(=O)NO)nc2cc(N(CCCl)CCCl)ccc21]

25. The combination according to claim 24, wherein the pharmacologically acceptable salt of the compound of formula I is the hydrochloride, hydrobromide, hydroiodide, sulfate, bisulfate, sulfamate, nitrate, phosphate, citrate, methanesulfonate, trifluoroacetate, glutamate, glucuronate, glutarate, malate, maleate, oxalate, succinate, fumarate, tartrate, tosylate, mandelate, salicylate, lactate, p-toluenesulfonate, naphthalenesulfonate or acetate, preferably acetate.

26. The combination according to claim 24, wherein the compound of formula I or a pharmaceutically acceptable salt thereof and the Brentuximab Vedotin are adapted for administration concurrently, sequentially or separately.

27. The combination according to any one of claims 24 to 26, wherein the molar ratio of the compound of formula I or a pharmaceutically acceptable salt thereof to Brentuximab Vedotin is from 1:0.1 to 1:5.

28. The combination according to any one of claims 24 to 26, wherein the molar ratio of the compound of formula I or a pharmaceutically acceptable salt thereof to Brentuximab Vedotin is from 1:0.25 to 1:2.

29. The combination according to any one of claims 24 to 28, wherein the compound of formula I or a pharmaceutically acceptable salt thereof and BrentuximabVedotin is a synergistic combination.

30. A kit comprising a combination according to any one of claims 24 to 29 and, optionally, instructions for treating a patient.

31. The combination according to any one of claims 24 to 29, for use in therapy.

32. A combination according to any one of claims 24 to 29 for use in a method of treating Hodgkin lymphoma in a patient in need thereof.

33. The combination according to claim 32, wherein the pharmacologically acceptable salt of the compound of formula I is the hydrochloride, hydrobromide, hydroiodide, sulfate, bisulfate, sulfamate, nitrate, phosphate, citrate, methanesulfonate, trifluoroacetate, glutamate, glucuronate, glutarate, malate, maleate, oxalate, succinate, fumarate, tartrate, tosylate, mandelate, salicylate, lactate, p-toluenesulfonate, naphthalenesulfonate or acetate.

34. The combination according to claim 32, wherein the pharmacologically acceptable salt of the compound of formula I is the acetate.

35. The combination according to any one of claims 32 to 34, wherein the molar ratio of the compound of formula I or a pharmaceutically acceptable salt thereof to Brentuximab Vedotin is from 1:0.1 to 1:5.

36. The combination according to any one of claims 32 to 34, wherein the molar ratio of the compound of formula I or a pharmaceutically acceptable salt thereof to Brentuximab Vedotin is from 1:0.25 to 1:2.

37. The combination according to any one of claims 32 to 35, wherein the compound of formula I or a pharmaceutically acceptable salt thereof and Brentuximab Vedotin is a synergistic combination.

38. The combination according to any one of claims 32 to 37, wherein the Hodgkin lymphoma is classical Hodgkin lymphoma or lymphocyte predominant Hodgkin lymphoma.

39. The combination according to any one of claims 32 to 37, wherein the Hodgkin lymphoma is nodular sclerosis classical Hodgkin lymphoma, mixed cellularity classical Hodgkin lymphoma, lymphocyte-depletion classical Hodgkin lymphoma or lymphocyte-rich classical Hodgkin lymphoma.

40. The combination according to any one of claims 32 to 36, wherein the Hodgkin lymphoma is nodular lymphocyte predominant Hodgkin lymphoma.

41. The combination according to any one of claims 32 to 40, wherein the Hodgkin lymphoma is relapsed/refractory Hodgkin lymphoma.

42. The combination according to any one of claims 32 to 41, wherein the compound of formula I or a pharmacologically acceptable salt thereof is administered intravenously to the patient in need thereof at a dosage level of from 1.5 to 150 mg/m² body surface area of the patient and Brentuximab Vedotin is administered intravenously to the patient in need thereof at a dosage level of from 0.5 mg/m² to 150 mg/m² body surface area of the patient.

43. The combination according to any one of claims 32 to 41, wherein the compound of formula I or a pharmacologically acceptable salt thereof is administered intravenously to the patient in need thereof at a dosage level of from 20 mg/m² to 80 mg/m² body surface area of the patient body surface area of the patient and Brentuximab Vedotin is administered intravenously to the patient in need thereof at a dosage level of from 5 mg/m² to 100 mg/m² body surface area of the patient.

44. The combination according to any one of claims 32 to 41, wherein the compound of formula I or a pharmacologically acceptable salt thereof is administered intravenously to the patient in need thereof at a dosage level of 40 mg/m² body surface area of the patient and Brentuximab Vedotin is administered intravenously to the patient in need thereof at a dosage level of 10 mg/m² of the patient.

45. The combination according to any one of claims 32 to 41, wherein the compound of formula I or a pharmacologically acceptable salt thereof is administered intravenously to the patient in need thereof at a dosage level of 40 mg/m² body surface area of the patient and Brentuximab Vedotin is administered intravenously to the patient in need thereof at a dosage level of 80 mg/m² of the patient.

46. The combination according to any one of claims 32 to 45, wherein the compound of formula I or a pharmaceutically acceptable salt thereof and Brentuximab Vedotin are administered concurrently, sequentially or separately.

47. The combination according to any one of claims 31 to 45, wherein the compound of formula I or a pharmacologically acceptable salt thereof and Brentuximab Vedotin are administered separately over a period of 2 to 3 hours intravenously to the patient in need thereof on days 1, 8 and 15 of a treatment cycle, for 4 to 6 weeks, followed by a break of 1 to 3 weeks.

48. The combination according to any one of claims 31 to 45, wherein the compound of formula I or a pharmacologically acceptable salt thereof and Brentuximab Vedotin are administered separately over a period of 2 to 3 hours intravenously to the patient in need thereof on days 1 and 22 of the treatment cycle, for 3 to 12 cycles of treatment, preferably 5 to 8 cycles, and most preferably 6 cycles.

49. The combination according to any one of claims 32 to 46, wherein the compound of formula I or a pharmacologically acceptable salt thereof and Brentuximab Vedotin are administered separately over a period of 2 to 3 hours intravenously to the patient in need thereof on days 1 and 8 of the treatment cycle, and this is repeated for 4 cycles.

50. The combination according to any one of claims 32 to 46, wherein the compound of formula I or a pharmacologically acceptable salt thereof and Brentuximab Vedotin are administered separately over a period of 2 to 3 hours intravenously to the patient in need thereof on days 1 and 15 of the treatment cycle, and this is repeated for 4 cycles.

51. The combination according to any one of claims 32 to 50, wherein the compound of the compound of formula I or a pharmacologically acceptable salt thereof and Brentuximab Vedotin are administered intravenously to the patient in need thereof, wherein the compound of formula I or a pharmacologically acceptable salt thereof is administered intravenously at a dosage level of from 20 mg/m² to 80 mg/m² body surface area of the patient and Brentuximab Vedotin is administered intravenously to the patient in need thereof at a dosage level of from 5 mg/m² to 100 mg/m² body surface area of the patient on days 1, 8 and 15 of a treatment cycle, wherein the compound of formula I or a pharmaceutically acceptable salt thereof and Brentuximab Vedotin are administered separately over a period of 2 to 3 hours, wherein the administration cycle is performed over a period of 4 to 6 weeks, followed by a break of 1 to 3 weeks.

52. The combination according to any one of claims 32 to 50, wherein the compound of the compound of formula I or a pharmacologically acceptable salt thereof and Brentuximab Vedotin are administered intravenously to the patient in need thereof, wherein the compound of formula I or a pharmacologically acceptable salt thereof is administered intravenously at a dosage level of from 20 mg/m² to 80 mg/m² body surface area of the patient and Brentuximab Vedotin is administered intravenously to the patient in need thereof at a dosage level of from 5 mg/m² to 100 mg/m² body surface area of the patient on days 1 and 22 of a treatment cycle, wherein the compound of formula I or a pharmaceutically acceptable salt thereof and Brentuximab Vedotin are administered separately over a period of 2 to 3 hours, wherein the administration cycle is performed over 3-12 consecutive cycles, more preferably 5-8 cycles and most preferably 6 cycles.

53. The combination according to any one of claims 32 to 50, wherein the compound of formula I or a pharmacologically acceptable salt thereof and Brentuximab Vedotin are administered intravenously to the patient in need thereof, wherein the compound of formula I or a pharmacologically acceptable salt thereof is administered intravenously at a dosage level of from 20 mg/m² to 80 mg/m² body surface area of the patient and Brentuximab Vedotin is administered intravenously to the patient in need thereof at a dosage level of from 5 mg/m² to 100 mg/m² body surface area of the patient, wherein the compound of formula I or a pharmaceutically acceptable salt thereof and Brentuximab Vedotin are administered separately over a period of 2 to 3 hours, wherein the administration cycle is performed on days 1 and 15 of a treatment cycle, for 4 consecutive cycles.

54. The combination according to any one of claims 32 to 50, wherein the compound of formula I or a pharmacologically acceptable salt thereof and Brentuximab Vedotin are administered intravenously to the patient in need thereof, wherein the compound of formula I or a pharmacologically acceptable salt thereof is administered intravenously at a dosage level of from 20 mg/m² to 80 mg/m² body surface area of the patient and Brentuximab Vedotin is administered intravenously to the patient in need thereof at a dosage level of from 5 mg/m² to 100 mg/m² body surface area of the patient, wherein the compound of formula I or a pharmaceutically acceptable salt thereof and Brentuximab Vedotin are administered separately over a period of 2 to 3 hours, wherein the administration cycle is performed on days 1 and 8 of a treatment cycle, for 4 consecutive cycles.

55. The combination according to any one of claims 32 to 54, wherein the patient in need thereof is given radiotherapy prior to or after treatment of the Hodgkin lymphoma with the compound of formula I or a pharmacologically acceptable salt thereof and Brentuximab Vedotin.

56. The combination according to claim 55, wherein said radiotherapy treatment is given to the patient in need thereof at a dose of 1 to 5 Gy over 5 consecutive days, and preferably 2 Gy over 5 consecutive days.

57. The use of a combination according to any one of claims 24 to 29 in the manufacture of a medicament for the treatment of Hodgkin lymphoma in a patient in need thereof.
